SEQUENCE LISTING (2 sequences)

SEQ ID NO: 1 - protein, 738 aa, Homo sapiens
CAGGTGCAGCTGGTGGAGTCTGGGGGAGGCTTGGTACAGCCTGGGGGGTCCCTGAGACTCTCCTGTGCAGCCTCTGGATTCACCTTTCGCAGCTATGCCATGAGCTGGGTCCGCCAGGCTCCAGGGAAGGGGCTGGAGTGGGTCTCAGCTATTAGTGGTCGTGGTGATAACACATACTACGCAGACTCCGTGAAGGGCCGGTTCACCATCTCCAGAGACAATTCCAAGAACACGCTGTATCTGCAAATGAACAGCCTGAGAGCCGAGGACACGGCCGTTTATTACTGTGCGAAAATGACAAGTAACGCGTTCGCATTTGACTACTGGGGCCAGGGAACCCTGGTCACCGTCTCCTCAGGTGGAGGCGGTTCAGGCGGAGGTGGCTCTGGCGGTGGCGGATCGCAGTCTGTGTTGACGCAGCCGCCCTCAGTGTCTGGGGCCCCAGGGCAGAGGGTCACCATCTCCTGCACTGGGAGCAGCTCCAACATCGGGGCAGGTTATGGTGTACACTGGTACCAGCAGCTTCCAGGAACAGCCCCCAAACTCCTCATCTATGGTAACACCAATCGGCCCTCAGGGGTCCCTGACCGATTCTCTGGCTTCAAGTCTGGCACCTCAGCCTCCCTGGCCATCACTGGGCTCCAGGCTGAGGATGAGGCTGATTATTACTGCCAGTCCTATGACAGCAGCCTGAGTGGTTGGGTGTTCGGCGGAGGGACCAAGCTGACCGTCCTAGGT

SEQ ID NO: 2 - protein, 246 aa, Homo sapiens
QVQLVESGGGLVQPGGSLRLSCAASGFTFRSYAMSWVRQAPGKGLEWVSAISGRGDNTYYADSVKGRFTISRDNSKNTLYLQMNSLRAEDTAVYYCAKMTSNAFAFDYWGQGTLVTVSSGGGGSGGGGSGGGGSQSVLTQPPSVSGAPGQRVTISCTGSSSNIGAGYGVHWYQQLPGTAPKLLIYGNTNRPSGVPDRFSGFKSGTSASLAITGLQAEDEADYYCQSYDSSLSGWVFGGGTKLTVLG

(19) United States
(12) Patent Application Publication
(10) Pub. No.: US 2006/0269542 A1
(43) Pub. Date: Nov. 30, 2006

(54) Immunoliposome composition for targeting to a HER2 cell receptor

(76) Inventors: Kristen Hjortsvang, Fremont, CA (US); Luke Guo, Lafayette, CA (US); Frances M.P. Wong, Smithfield, RI (US); Azar Najafi, Danville, CA (US); Anthony Huang, Saratoga, CA (US); Robert Abra, San Francisco, CA (US); Laura Kelley, Santa Cruz, CA (US)

Correspondence Address: PERKINS COIE LLP, P.O. BOX 2168, MENLO PARK, CA, 94026, US

(21) Appl. No.: 11/408,441

(22) Filed: Apr. 20, 2006

Related U.S. Application Data
(60) Provisional application No. 60/736,669, filed on Nov. 14, 2005.
(60) Provisional application No. 60/674,029, filed on Apr. 22, 2005.

(51) Int. Cl. A61K 39/395; A61K 9/127

(52) U.S. Cl. 424/133.1; 424/450

(57) ABSTRACT

An immunoliposome composition comprised of liposomes bearing a ligand for targeting to cells expressing a growth factor receptor, such as HER2, is described. Binding of the immunoliposome to HER2-expressing cells results in internalization of the immunoliposome for cytoplasmic delivery of an entrapped drug.

### CROSS-REFERENCE TO RELATED APPLICATIONS

This application claims the benefit of U.S. Provisional Application No. 60/736,669, filed Nov. 14, 2005 and to U.S. Provisional Application No. 60/674,029, filed Apr. 22, 2005, both incorporated herein by reference in their entirety.

### TECHNICAL FIELD

The present subject matter relates to a liposome composition. In particular, the subject matter relates to liposomes targeted to a specific cell receptor for delivery of a liposome-entrapped drug to the cell.

### BACKGROUND

Liposomes are spherical vesicles comprised of concentrically ordered lipid bilayers that encapsulate an aqueous phase. Liposomes serve as a delivery vehicle for therapeutic agents contained in the aqueous phase or in the lipid bilayers. Delivery of drugs in liposome-entrapped form can provide a variety of advantages, depending on the drug, including, for example, a decreased drug toxicity, altered pharmacokinetics, or improved drug solubility. Liposomes when formulated to include a surface coating of hydrophilic polymer chains, so-called Stealth® or long-circulating liposomes, offer the further advantage of a long blood circulation lifetime, due in part to reduced removal of the liposomes by the mononuclear phagocyte system. Often an extended lifetime is necessary in order for the liposomes to reach their desired target region or cell from the site of injection.

Targeted liposomes have targeting ligands or affinity moieties attached to the surface of the liposomes. The targeting ligands may be antibodies or fragments thereof, in which case the liposomes are referred to as immunoliposomes. When administered systemically targeted liposomes deliver the entrapped therapeutic agent to a target tissue, region or, cell. Because targeted liposomes are directed to a specific region or cell, healthy tissue is not exposed to the therapeutic agent. Such targeting ligands can be attached directly to the liposomes' surfaces by covalent coupling of the targeting ligand to the polar head group residues of liposomal lipid components (see, for example, U.S. Pat. No. 5,013,556). This approach, however, is suitable primarily for liposomes that lack surface-bound polymer chains, as the polymer chains interfere with interaction between the targeting ligand and its intended target (Klibanov, A. L., et al., Biochim. Biophys. Acta., 1062:142-148 (1991); Hansen, C. B., et al., Biochim. Biophys. Acta, 1239:133-144 (1995)).

Alternatively, the targeting ligands can be attached to the free ends of the polymer chains forming the surface coat on the liposomes (Allen. T. M., et al., Biochim. Biophys. Acta, 1237:99-108 (1995); Blume, G. et al., Biochim. Biophys. Acta, 1149:180-184 (1993)). In this approach, the targeting ligand is exposed and readily available for interaction with the intended target.

The HER2 (c-erbB2, neu) protonocogene and p185^{HER2}, the growth factor receptor-tyrosine kinase it encodes, appear to play a role in the pathogenesis of many human cancers. Overexpression of p185^{HER2} occurs in 20-30% of breast cancers, and predicts a poor prognosis for these patients (Park, J. W. et al., Proc. Natl. Acad. Sci. USA, 92:1327 (1995)).

The p185^{HER2} receptor is an attractive target for an antibody based therapy, since when present, P185^{HER2} over-expression generally occurs homogenously within primary breast tumors, yet is expressed only at low levels in certain normal epithelial cells. A murine anti-p185^{HER2} monoclonal antibody, muAb4D5, and a humanized version of this antibody, trastuzumab, have been developed (Carter, P. et al., Proc. Natl. Acad. Sci. USA, 89:4285 (1992); U.S. Pat. No. 5,677,171). Various antibodies that bind to P185^{HER2} have also been described (WO 99/55367).

Liposomes bearing an antibody specific for the HER2 receptor have been reported (Park, J. W. et al., Proc. Natl. Acad. Sci. USA, 92:1327 (1995); Park, J. W. et al., J. Controlled Release, 74:95 (2001); Nielsen, U. B. et al., Biochim. Biophys. Acta, 1591:109 (2002); U.S. Pat. No. 6,214,388). An apparent relationship between the number of antibodies per liposome, i.e., antibody density, on the extent of cellular binding and uptake is disclosed (Nielsen, U. B. et al., Biochim. Biophys. Acta, 1591:109 (2002)). The results of that study indicate that varying the density of antibodies on the liposome surface from 0 to 30 corresponds to an increase in cellular uptake, which reaches a maximum at 30 antibodies per liposome. Increasing the number of anti-HER2 antibodies from 30 to 100 per liposome did not increase cellular uptake of the liposomes.

The effectiveness of a cancer treatment is directly related to the treatment's ability to target and to kill the cancer cells while affecting as few healthy cells as possible. While the concept of targeting a drug specifically to a tumor cell has been widely discussed, there remains a need for a formulation that is highly selective for certain cancer cells and that is designed for optimal in vivo binding to such certain cancer cells.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

In one aspect, a composition is described, the composition comprising liposomes comprised of (i) vesicle-forming lipids; (ii) a lipopolymer; (iii) an anti-HER2 receptor antibody conjugate comprised of a hydrophobic moiety a hydrophilic polymer; and (iv) an entrapped drug is described. The amount of conjugate is present in an amount effective to provide greater than about 2 antibodies per liposome, on average, and fewer than about 25 antibodies per liposome, on average.

In one embodiment, the antibody has a molecular weight of between 5,000-50,000 Daltons, more preferably of between 10,000-50,000, and still more preferably of between 10,000-30,000. In another embodiment, the antibody has a molecular weight of less than 100,000 Daltons, more preferably of less than about 35,000 Daltons, and still more preferably of less than 30,000 Daltons.

In another embodiment, the antibody has an amino acid sequence that has at least about 80%, preferably at least about 85%, more preferably at least about 90%, sequence identity with SEQ ID NO:2.

In another embodiment, the hydrophilic polymer is polyethylene glycol having a molecular weight of between 750-5000 Daltons.

In another embodiment, the entrapped drug is a cytotoxic drug. In yet another embodiment, the entrapped drug is an anti-tumor agent. An exemplary entrapped drug is anthracycline, such as doxorubicin.

In another embodiment, the amount of conjugate provides fewer than about 20 antibodies per liposome, on average, 48 hours after in vivo administration.

In another aspect, an immunoliposome formulation is provided, the formulation, comprising liposomes comprised of (i) at least one rigid vesicle-forming lipid; (ii) a lipopolymer comprised of a hydrophobic moiety and polyethylene glycol; (iii) a conjugate comprised of a hydrophobic moiety, polyethylene glycol, and an anti-Her-2 receptor single chain antibody having at least 80% identity to SEQ ID NO:2; and (iv) an entrapped drug having anti-tumor activity. The liposomes are characterized by an amount of conjugate effective to provide greater than about 2 and fewer than about 15 antibodies per liposome 96 hours after in vivo administration.

In one embodiment, the rigid vesicle-forming lipid is hydrogenated soy phosphatidylcholine.

In another embodiment, the liposomes further comprise cholesterol.

In still another aspect, an immunoliposome formulation is described, the formulation, comprising liposomes comprised of (i) at least one rigid vesicle-forming lipid; (ii) a lipopolymer comprised of a hydrophobic moiety and polyethylene glycol; (iii) a conjugate comprised of a hydrophobic moiety, polyethylene glycol, and an anti-Her-2 receptor single chain antibody having the identity of SEQ ID NO:2; and (iv) an entrapped drug having anti-tumor activity. The immunoliposome formulation when administered in vivo provides an area under the curve that is greater than or not more than 25% lower than the area under the curve of liposomes comprised of like components but lacking the antibody.

In one embodiment, the amount of conjugate provides fewer than about 20 antibodies per liposome, on average, 48 hours after in vivo administration. In another embodiment, the amount of conjugate provides fewer than about 15 antibodies per liposome, on average, 96 hours after in vivo administration.

In yet another embodiment, the amount of conjugate provides greater than two antibodies per liposome, on average, 48 hours after in vivo administration and less than about 20 antibodies per liposome, on average, 48 hours after in vivo administration.

In yet another aspect, an immunoliposome formulation is described, where the formulation comprises liposomes comprised of (i) at least one rigid vesicle-forming lipid; (ii) optionally, a lipopolymer comprised of a hydrophobic moiety and polyethylene glycol; (iii) a conjugate comprised of a hydrophobic moiety, polyethylene glycol, and an anti-Her-2 receptor single chain antibody having at least 80% identity to SEQ ID NO:2; and (iv) an entrapped drug having anti-tumor activity. The composition is characterized by the feature that 96 hours after administration of the immunoliposomes, between 30-60% of antibodies dissociate from each liposome to provide a composition, 96 hours after in vivo administration that has fewer than about 25 antibodies per liposome.

In still another aspect, a liposome composition prepared according to a certain process is described, the process being comprised of (a) providing liposomes, optionally having an outer coating of hydrophilic polymer chains and/or an entrapped drug; (b) incubating the liposomes with an amount of conjugate comprised of a hydrophobic moiety, polyethylene glycol, and an anti-Her2 receptor single chain antibody having at least 80% identity to SEQ ID NO:2; and the amount of conjugate being selected to provide (i) more than two antibodies per liposome on average 96 hours after in vivo administration; (ii) fewer than 150 antibodies per liposome on average; and/or (iii) fewer than about 15 antibodies per liposome on average 96 hours after in vivo administration.

In one embodiment, the amount of conjugate provides 12 or fewer, alternatively 10 or fewer, alternatively 8 or fewer, antibodies per liposome on average.

In another aspect, a composition is described, the composition comprising liposomes as described above, but where the liposomes prior to in vivo administration have fewer than 25 antibodies per liposome and after in vivo administration, the liposomes lose between about 20-50% of the antibodies yet retain binding to the Her-2 receptor sufficient for cytotoxicity.

In still another aspect, a method of preparing an immunoliposome composition is provided. The method comprises providing immunoliposomes comprised of (i) vesicle-forming lipids; (ii) optionally, a lipopolymer; (iii) a conjugate comprised of a hydrophobic moiety, a hydrophilic polymer, and an antibody having binding affinity for an target, such as an extracellular domain of a Her-2 receptor; and (iv) an entrapped drug. The conjugate is included in the composition in a first amount sufficient to provide a first selected number of antibodies per liposome. The liposomes are brought in to contact with blood, in vitro or in vivo, and the number of antibodies per liposome at one or more time points upon contact of the liposomes with blood is determined, for example by a suitable analytical technique such as chromatography. Based on the determination of the number of antibodies at a first selected time point after contact with blood, a second amount of conjugate sufficient to provide a second, higher number of antibodies per liposome in order to provide at least two antibodies per liposome after contact with blood at such a time point is selected.

In one embodiment, the method includes selecting a second amount of conjugate effective to provide fewer than 50 antibodies per liposome. In another embodiment, a second amount of conjugate effective to provide 30 or fewer antibodies per liposome is selected.

In another embodiment, the method includes providing liposomes having a first amount of conjugate that provides fewer than 150 antibodies per liposome, more preferably 100 or fewer antibodies per liposome, and still more preferably 75 or fewer antibodies per liposome.

In addition to the exemplary aspects and embodiments described above, further aspects and embodiments will become apparent by reference to the drawings and by study of the following descriptions.

### BRIEF DESCRIPTION OF THE DRAWINGS

FIGS. 1A-1B show a synthetic reaction scheme for preparation of a lipid-polymer-antibody conjugate where a maleimide of a DSPE carbamate of polyethylene glycol (PEG) bis (amine) is formed;

FIG. 2A shows the cell viability, expressed as a percent of untreated control cells, as a function of doxorubicin concentration, in μg/mL, after 10 minutes of exposure to doxorubicin administered as free drug (triangles), entrapped in liposomes (circles), or entrapped in immunoliposomes containing 2 (circles), 5 (squares), 7.5 (diamonds), or 15 (triangles) antibodiess per liposome;

FIG. 2B shows the cell viability, expressed as a percent of untreated control cells, as a function of doxorubicin concentration, in μg/mL, after four hours of exposure to doxorubicin administered as free drug (diamonds), entrapped in liposomes (triangles), or entrapped in immunoliposomes containing 7.5 (X symbols), 15 (* symbols), 30 (circles), or 45 (squares) antibodies per liposome;

FIGS. 3A-3H show the elution profiles of radiolabeled (¹²⁵I) scFv from immunoliposomes having 15 scFv antibodies per liposome from aliquots removed during incubation of the immunoliposomes in human plasma, the aliquots removed at times of 0 hours (FIG. 3A), 1 hour (FIG. 3B), 4 hours (FIG. 3C), 8 hours (FIG. 3D), 24 hours (FIG. 3E), 48 hours (FIG. 3F), 72 hours (FIG. 3G), and 96 hours (FIG. 3E);

FIG. 4A shows the dissociation of ¹²⁵I labeled scFv antibody from immunoliposome formulations having antibody/liposome ratios of 7.5:1 (diamonds), 15:1 (squares), 30:1 (circles), 45:1 (triangles), and 90:1 (*) as a function of incubation time, in hours, in human plasma in vitro;

FIG. 4B shows the percent of ¹²⁵I-label remaining in the immunoliposomes having antibody/liposome ratios of 7.5:1 (diamonds), 15:1 (squares), 30:1 (circles), 45:1 (triangles), and 90:1 (*) as a function of incubation time, in hours, in human plasma in vitro;

FIG. 5A shows the percent of radiolabeled antibody dissociated from the immunoliposome formulation having 15 antibodies per liposome in two different studies (diamonds, squares) as a function of incubation time, in hours, in human plasma in vitro;

FIG. 5B shows the percent of radiolabeled antibody remaining in the immunoliposomes (diamonds) and dissociated from the immunoliposomes (squares) from the immunoliposome formulation having 15 antibodies per liposome as a function of incubation time, in hours, in human plasma;

FIG. 6A shows the percentage of ¹²⁵I-labeled scFv antibody recovered in the liposomal fraction of plasma samples as a function of time, in hours, after administration of a 15:1 immunoliposome formulation to rats;

FIG. 6B shows the concentration of ¹²⁵I-labeled scFv antibody, in ng/mL, recovered in the liposomal fraction of rat plasma after separation on the Sepharose CL-4B column as a function of time, in hours, after administration of a 15:1 immunoliposome formulation to rats;

FIG. 6C shows the concentration of ¹²⁵I-labeled scFv antibody, in ng/mL, recovered in the free or plasma fraction of rat plasma after separatioin on a Sepharose CL-4B column as a function of time, in hours, after administration of a 15:1 immunoliposome formulation to rats;

FIG. 7A shows the percentage of ¹²⁵I-labeled scFv remaining in the plasma (diamonds), in the blood (closed squares) and the percentage of doxorubicin in the plasma (triangles) as a function of time, in hours, after administration of a 15:1 immunoliposome formulation to rats. Also shown is the percentage of ¹²⁵I-labeled scFv antibody in the plasma (open circles) and in the blood (open squares) as a function of time, in hours, after administration as a free conjugate to rats at a doxorubicin dose of 2 mg/kg;

FIG. 7B shows the plasma concentration of doxorubicin, in μg/mL, as a function of time, in hours, after administration of a 15:1 immunoliposome formulation to rats at a dose of 2 mg/kg;

FIG. 8 is a plot of the ratio of ¹²⁵I-labeled scFv antibody to doxorubicin in the blood, in ng/μg, as a function of time, in hours, after administration of a 15:1 immunoliposome formulation to rats;

FIG. 9 is a plot of plasma doxorubicin concentration, in μg/mL, as a function of time, in hours, after in vivo administration of PEGylated liposomes containing doxorubicin (diamonds) or of immunoliposomes containing 15 (squares), 75 (triangles), 150 (x) or 300 (*) scFv antibodies per liposome;

FIGS. 10A-10B shows the tumor volume, in percent relative to the initial tumor size, (FIG. 10A) and the percent change in body weight (FIG. 10B) in mice bearing a breast tumor xenograft and treated with saline (open circles), PEGylated liposomes containing doxorubicin (open squares) or immunoliposomes containing 7.5 (diamonds), 15 (triangles), 30 (closed squares), or 45 (closed circles) scFv antibodies per liposome;

FIG. 11 is a plot of relative tumor volume, taken as a percent of initial tumor volume, as a function of time, in days, in mice bearing a breast tumor xenograft and treated with saline (open circles), PEGylated liposomes containing doxorubicin at dosages of 2 mg/kg (open squares) and 3 mg/kg (open diamonds) or with a 15:1 immunoliposome formulation at dosages of 2 mg/kg (closed squares), 3 mg/kg (closed diamonds) or 4 mg/kg (closed triangles);

FIG. 12 is a graph of plasma doxorubicin concentration, in ng/mL, as a function of time, in hours, after intravenous administration to monkeys of doxorubicin (10 mg/mL) entrapped in immunoliposomes bearing 15 scFv antibodies per liposome, on average, (circles) or entrapped in PEGylated liposomes (squares);

FIG. 13A is a graph of the plasma doxorubicin concentration, in ng/mL, as a function of time, in hours, after intravenous administration to monkeys of doxorubicin entrapped in immunoliposomes bearing 15 scFv antibodies per liposome, on average, the doxorubicin administered at dosages of 1 mg/kg (circles), 5 mg/kg (squares), and 10 mg/kg (triangles);

FIG. 13B is a graph of the plasma antibody concentration, in ng/mL, as a function of time, in hours, after intravenous administration to monkeys of doxorubicin entrapped in immunoliposomes bearing 15 scFv antibodies per liposome, on average, the immunoliposomes administered at doxorubicin dosages of 1 mg/kg (circles), 5 mg/kg (squares), and 10 mg/kg (triangles);

FIG. 14A is a plot of the ratio of scFv antibody/doxorubicin concentration in plasma, in ng/μg, as a function of time, in hours, after administration to monkeys of doxorubicin entrapped in immunoliposomes bearing 15 scFv antibodies per liposome, on average, the immunoliposomes administered at doxorubicin dosages of 1 mg/kg (diamonds), 5 mg/kg (squares), and 10 mg/kg (triangles);

FIG. 14B is a plot of the ratio of scFv antibody/doxorubicin concentration in plasma normalized to the initial antibody/doxorubicin ratio, as a function of time, in hours, after administration to monkeys of doxorubicin entrapped in immunoliposomes bearing 15 scFv antibodies per liposome, on average, the immunoliposomes administered at doxorubicin dosages of 1 mg/kg (diamonds), 5 mg/kg (squares), and 10 mg/kg (triangles); and

FIG. 15A is a plot of doxorubicin concentration, in ng/mL, as a function of time, in hours, after administration to monkeys of doxorubicin entrapped in immunoliposomes bearing 15 scFv antibodies per liposome, on average, the immunoliposomes administered at doxorubicin dosages of 0.5 mg/kg (squares), 2 mg/kg (triangles), and 4 mg/kg (inverted triangles);

FIG. 15B is a plot of doxorubicin concentration, in ng/mL, as a function of time, in hours, after administration to monkeys of doxorubicin entrapped in immunoliposomes bearing 15 scFv antibodies per liposome, on average, the immunoliposomes administered at a doxorubicin dosage of 4 mg/kg six times over a six month period, the data corresponding to the first (inverted triangles) and sixth (squares) doses;

FIG. 15C is a plot of doxorubicin concentration, in ng/mL, as a function of time, in hours, after administration to monkeys of 4 mg/kg doxorubicin in free form (triangles), entrapped in PEGylated liposomes (DOXIL®; squares), or entrapped in mmunoliposomes bearing 15 scFv antibodies per liposome, on average, (inverted triangles).

### BRIEF DESCRIPTION OF THE SEQUENCES

SEQ ID NO:1 is the nucleotide sequence of an antibody having binding affinity for the extracellular domain of c-erb-B2 receptor, also referred to herein as the HER2 receptor and the p185HER2 receptor.

SEQ ID NO: 2 is the amino acid sequence of a single chain antibody (scFv) designated F5, having the ability to specifically bind the extracellular domain of HER2 receptor.

### DETAILED DESCRIPTION

I. Definitions

Unless otherwise noted, the term “vesicle-forming lipid” refers to any lipid capable of forming part of a stable micelle or liposome composition and typically including one or two hydrophobic, hydrocarbon chains or a steroid group and may contain a chemically reactive group, such as an amine, acid, ester, aldehyde or alcohol, at its polar head group.

As used herein, an “antibody” includes whole antibodies and any antigen binding fragment or single chain thereof. Thus, the antibody includes any protein or peptide containing molecule that comprises at least a portion of an immunoglobulin molecule, such as but not limited to at least one complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The term “antibody” is further intended to encompass antibody digestion fragments, specified portions and variants thereof, including antibody mimetics or comprising domains of antibodies that mimic the structure and/or function of an antibody or specified fragment or portion thereof, including single chain antibodies and fragments thereof. Functional fragments include antigen-binding fragments that bind to a mammalian HER2 protein which is a growth factor receptor. Examples of binding fragments encompassed within the term “antigen binding portion” of an antibody include (i) a Fab fragment, a monovalent fragment consisting of the VL, VH, CL and CH, domains; (ii) a F(ab′)₂ fragment, a bivalent fragment comprising two Fab fragments linked by a disulfide bridge at the hinge region; (iii) a Fd fragment consisting of the VH and CH, domains; (iv) a Fv fragment consisting of the VL and VH domains of a single arm of an antibody, (v) a dAb fragment (Ward et al., Nature, 341:544-546 (1989)), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. Science, 242:423-426 (1988), Huston et al., Proc. Natl. Acad. Sci. USA, 85:5879-5883 (1988)). Such single chain antibodies are also intended to be encompassed within the term antibody. These antibodies are obtained using conventional techniques known to those with skill in the art, and the fragments are screened for utility in the same manner as are intact antibodies.

Such fragments can be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. For example, a combination gene encoding a F(ab′)₂ heavy chain portion can be designed to include DNA sequences encoding the CH₁ domain and/or hinge region of the heavy chain. The various portions of antibodies can be joined together chemically by conventional techniques, or can be prepared as a contiguous protein using genetic engineering techniques.

By “HER2 receptor” and “p185HER2” is meant the protein encoded by the HER2 or ERRB2 gene (v-erb-b2, erythroblastic leukemia viral oncogene homolog 2) and also called the neuro/glioblastoma derived oncogene homolog; avian erythroblastic leukemia viral (v-erb-b2) oncogene homolog 2; v-erb-b2 avian erythroblastic leukemia viral oncogene homolog 2 (neuro/glioblastoma derived oncogene homolog). The HER2/ERRB2 gene encodes a member of the epidermal growth factor (EGF) receptor family of receptor tyrosine kinases. The coding sequence for HER2 is given by the reference sequence NM_{—}004448 which translation product is given by NP_{—}004439. The HER2 receptor has no ligand binding domain of its own and therefore cannot bind growth factors. However, it does bind tightly to other ligand-bound EGF receptor family members to form a heterodimer, stabilizing ligand binding and enhancing kinase-mediated activation of downstream signalling pathways, such as those involving mitogen-activated protein kinase and phosphatidylinositol-3 kinase. Allelic variations at amino acid positions 654 and 655 of isoform a (positions 624 and 625 of isoform b) have been reported, with the most common allele, Ile654/Ile655. Alternative splicing results in several additional transcript variants, some encoding different isoforms. All allelic and splice variants are included in the meaning of “HER2 receptor”.

An “internalizing antibody” is an antibody that, upon binding to a receptor or other ligand on a cell surface, is transported into the cell, for example, into a lysozyme or other organelle or into the cytoplasm.

The term “isolated” refers to material which is substantially or essentially free from components that normally accompany it as found in its native state.

The terms “identical” or percent “identity” or percent “homology” in the context of two or more nucleic acid or polypeptide sequences, refer to two or more sequences or subsequences that are the same or have a specified percentage of amino acid residues or nucleotides that are the same, when compared and aligned for maximum correspondence, as measured by visual inspection or using a computer algorithm.

As used herein, the term “affinity” of an antibody refers the dissociation constant, K_{D}, the antibody for a predetermined antigen. High affinity antibodies have a K_{D} of 10⁻⁸ M or less, more preferably 10⁻⁹ M or less and even more preferably 10⁻¹⁰ M or less, for a predetermined antigen. The term “Kdis” or “K_{D},” or “Kd’ as used herein, is intended to refer to the dissociation rate of a particular antibody-antigen interaction. The “K_{D}”, is the ratio of the rate of dissociation (k₂), also called the “off-rate (k_{off})”, to the rate of association rate (k₁) or “on-rate (kₒₙ)”. Thus, K_{D} equals k2/k1 or k_{off}/kₒₙ and is expressed as a molar concentration (M). It follows that the smaller K_{D}, the stronger the binding. So a K_{D} of 10⁻⁶M (or 1 μM) indicates weak binding compared to 10⁻⁹ M (or 1 nM).

The phrases “an antibody recognizing an antigen” and “an antibody specific for an antigen” are used interchangeably herein with the term “an antibody which binds specifically to an antigen”. As used herein, “specific binding” and “specifically binds” refers to antibody binding to a predetermined antigen with greater affinity than for other antigens or proteins. Typically, the antibody binds with a dissociation constant (K_{D}) of 10⁻⁶ M or less, and binds to the predetermined antigen with a K_{D} that is at least twofold less than its K_{D} for binding to a non-specific antigen (e.g., BSA, casein, or any other specified polypeptide) other than the predetermined antigen. The phrases “an antibody recognizing an antigen” and “an antibody specific for an antigen” are used interchangeably herein with the term “an antibody which binds specifically to an antigen”.

As disclosed and claimed herein, the sequence set forth in SEQ ID NO: 2 include “conservative sequence modifications”, i.e., nucleotide and amino acid sequence modifications which do not significantly affect or alter the binding characteristics of the antibody encoded by the nucleotide sequence or containing the amino acid sequence. Such conservative sequence modifications include nucleotide and amino acid substitutions, additions and deletions. Modifications can be introduced into SEQ ID NO: 2 by standard techniques known in the art, such as site-directed mutagenesis and PCR-mediated mutagenesis. Conservative amino acid substitutions include ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

II. Liposome Composition

In one aspect, the invention relates to a liposome composition comprised of liposomes that include as a targeting ligand an antibody having binding specificity for a growth factor receptor HER2. The HER2 targeting ligand is incorporated into the liposomes in the form of a lipid-polymer-antibody conjugate, also referred to herein as a lipid-polymer-ligand conjugate. As will be described below, the antibody has specific affinity for the external domain of the HER2 receptor and targets the liposomes to cells that express HER2 receptor. The following sections describe the liposome components, including the liposome lipids and therapeutic agents, preparation of liposomes bearing an HER2 targeting ligand, and methods of using the liposomal composition for treatment of disorders.

A. Liposome Lipid Components

Liposomes suitable for use in the composition of the present invention include those composed primarily of vesicle-forming lipids. Such a vesicle-forming lipid is one which can form spontaneously into bilayer vesicles in water, as exemplified by the phospholipids, with its hydrophobic moiety in contact with the interior, hydrophobic region of the bilayer membrane, and its head group moiety oriented toward the exterior, polar surface of the membrane. Lipids capable of stable incorporation into lipid bilayers, such as cholesterol and its various analogs, can also be used in the liposomes.

The vesicle-forming lipids are preferably lipids having two hydrocarbon chains, typically acyl chains, and a head group, either polar or nonpolar. There are a variety of synthetic vesicle-forming lipids and naturally-occurring vesicle-forming lipids, including the phospholipids, such as phosphatidylcholine, phosphatidylethanolamine, phosphatidic acid, phosphatidylinositol, and sphingomyelin, where the two hydrocarbon chains are typically between about 14-22 carbon atoms in length, and have varying degrees of unsaturation. The above-described lipids and phospholipids whose carbon chains have varying degrees of saturation can be obtained commercially or prepared according to published methods. Other suitable lipids include glycolipids, cerebrosides and sterols, such as cholesterol.

Cationic lipids are also suitable for use in the liposomes of the invention, where the cationic lipid can be included as a minor component of the lipid composition or as a major or sole component. Such cationic lipids typically have a lipophilic moiety, such as a sterol, an acyl or diacyl chain, and where the lipid has an overall net positive charge. Preferably, the head group of the lipid carries the positive charge. Exemplary cationic lipids include 1,2-dioleyloxy-3-(trimethylamino) propane (DOTAP); N-[1-(2,3,-ditetradecyloxy)propyl]-N,N-dimethyl-N-hydroxyethylammonium bromide (DMRIE); N-[1-(2,3,-dioleyloxy)propyl]-N,N-dimethyl-N-hydroxy ethylammonium bromide (DORIE); N-[1-(2,3-dioleyloxy) propyl]-N,N,N-trimethylammonium chloride (DOTMA); 3 [N-(N′,N′-dimethylaminoethane) carbamoly]cholesterol (DC-Chol); and dimethyldioctadecylammonium (DDAB). The cationic vesicle-forming lipid may also be a neutral lipid, such as dioleoylphosphatidyl ethanolamine (DOPE) or an amphipathic lipid, such as a phospholipid, derivatized with a cationic lipid, such as polylysine or other polyamine lipids. For example, the neutral lipid (DOPE) can be derivatized with polylysine to form a cationic lipid.

The vesicle-forming lipid can be selected to achieve a specified degree of fluidity or rigidity, to control the stability of the liposome in serum, to control the conditions effective for insertion of the targeting conjugate, as will be described, and/or to control the rate of release of the entrapped agent in the liposome. Liposomes having a more rigid lipid bilayer, or a liquid crystalline bilayer, are achieved by incorporation of a relatively rigid lipid, e.g., a lipid having a relatively high phase transition temperature, e.g., up to 60° C. Rigid, i.e., saturated, lipids contribute to greater membrane rigidity in the lipid bilayer. Other lipid components, such as cholesterol, are also known to contribute to membrane rigidity in lipid bilayer structures.

On the other hand, lipid fluidity is achieved by incorporation of a relatively fluid lipid, typically one having a lipid phase with a relatively low liquid to liquid-crystalline phase transition temperature, e.g., at or below room temperature.

The liposomes also include a vesicle-forming lipid covalently attached to a hydrophilic polymer, also referred to herein as a “lipopolymer”. As has been described, for example in U.S. Pat. No. 5,013,556, including such a polymer-derivatized lipid in the liposome composition forms a surface coating of hydrophilic polymer chains around the liposome. The surface coating of hydrophilic polymer chains is effective to increase the in vivo blood circulation lifetime of the liposomes when compared to liposomes lacking such a coating.

Vesicle-forming lipids suitable for derivatization with a hydrophilic polymer include any of those lipids listed above, and, in particular phospholipids, such as distearoyl phosphatidylethanolamine (DSPE).

Hydrophilic polymers suitable for derivatization with a vesicle-forming lipid include polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol, polyaspartamide and hydrophilic peptide sequences. The polymers may be employed as homopolymers or as block or random copolymers.

A preferred hydrophilic polymer chain is polyethyleneglycol (PEG), preferably as a PEG chain having a molecular weight between 500-10,000 daltons, more preferably between 750-10,000 daltons, still more preferably between 750-5000 daltons. Methoxy or ethoxy-capped analogues of PEG are also preferred hydrophilic polymers, commercially available in a variety of polymer sizes, e.g., 120-20,000 Daltons.

Preparation of vesicle-forming lipids derivatized with hydrophilic polymers has been described, for example in U.S. Pat. No. 5,395,619. Preparation of liposomes including such derivatized lipids has also been described, where typically between 1-20 mole percent of such a derivatized lipid is included in the liposome formulation (see, for example, U.S. Pat. No. 5,013,556).

B. Targeting Antibody

The liposome composition also includes an antibody that targets the lipid particles to a cell. In one embodiment, the antibody binds specifically to HER2 receptor on the surface of a tumor derived cell. In another embodiment, the antibody comprises at least one binding domain which specifically binds the HER2 receptor on the surface of a tumor-derived cell. In an alternate embodiment, the antibody is a single chain antibody comprising at least one binding domain which specifically binds HER2 receptor on the surface of a tumor-derived cell.

In a preferred embodiment, the antibody for use in the liposome composition described herein is a single chain antibody comprising at least one binding domain which specifically binds HER2 receptor on the surface of a tumor-derived cell and has a sequence identified herein as SEQ ID NO: 2. Preparation of this antibody is described in WO 99/55367 and the antibody is designated F5. The antibody is a single chain antibody fragment (scFv) that specifically binds to the extracellular domain of the c-erb-B2 protein product of the HER2/neu oncogene, and is also referred to herein as the anti-HER2 antibody, or an antibody having binding affinity for the HER2 receptor. The F5 antibody is comprised of human antibody variable domains connected by a linker molecule and contains 251 amino acids with a terminal cysteine (molecular weight 27.6 kD), and has moderate HER2 binding affinity (K_{d}=150-300 nM or 1.5−3×10⁻⁷ M). The anti-HER2 antibody of the invention is rapidly internalized into cells that express the HER2 receptor on their membrane surface.

In one embodiment, the anti-HER2 antibody has a sequence that represents conservative substitution to SEQ ID NO: 2.

C. Preparation of Lipid-Polymer-Antibody Conjugate

As described above, the anti-HER2 antibody is covalently attached to the free distal end of a hydrophilic polymer chain, which is attached at its proximal end to a vesicle-forming lipid. There are a wide variety of techniques for attaching a selected hydrophilic polymer to a selected lipid and activating the free, unattached end of the polymer for reaction with a selected ligand, and in particular, the hydrophilic polymer polyethyleneglycol (PEG) has been widely studied (Allen, T. M., et al., Biochemicia et Biophysica Acta, 1237:99-108 (1995); Zalipsky, S., Bioconjugate Chem., 4(4):296-299 (1993); Zalipsky, S., et al. FEBS Lett., 353:71-74 (1994); Zalipsky, S. et al., Bioconjugate Chemistry, 6(6):705-708 (1995); Zalipsky, S., in STEALTH LIPOSOMES (D. Lasic and F. Martin, Eds.) Chapter 9, CRC Press, Boca Raton, Fla. (1995)).

Generally, the PEG chains are functionalized to contain reactive groups suitable for coupling with, for example, sulfhydryls, amino groups, and aldehydes or ketones (typically derived from mild oxidation of carbohydrate portions of an antibody) present in a wide variety of ligands. Examples of such PEG-terminal reactive groups include maleimide (for reaction with sulfhydryl groups), N-hydroxysuccinimide (NHS) or NHS-carbonate ester (for reaction with primary amines), hydrazide or hydrazine (for reaction with aldehydes or ketones), iodoacetyl (preferentially reactive with sulfhydryl groups) and dithiopyridine (thiol-reactive). Synthetic reaction schemes for activating PEG with such groups are set forth in U.S. Pat. Nos. 5,631,018, 5,527,528, 5,395,619, and the relevant sections describing synthetic reaction procedures are expressly incorporated herein by reference.

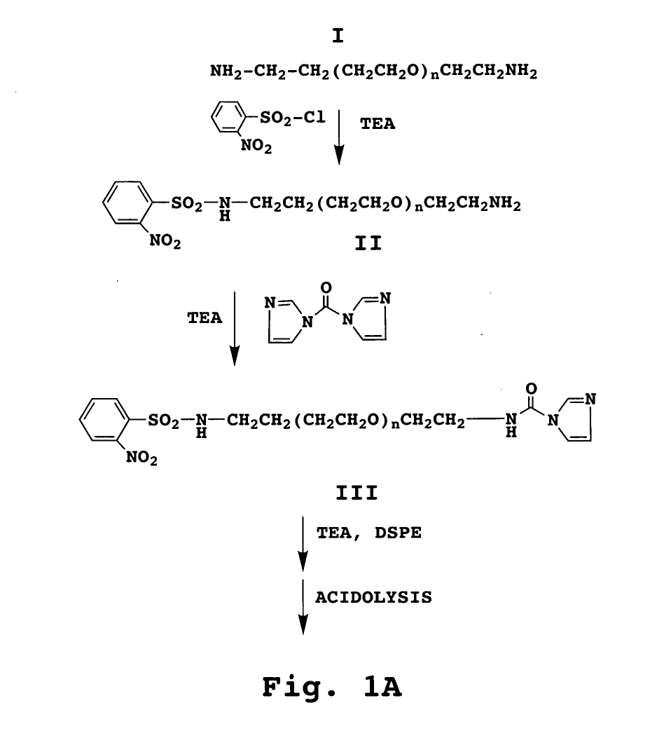

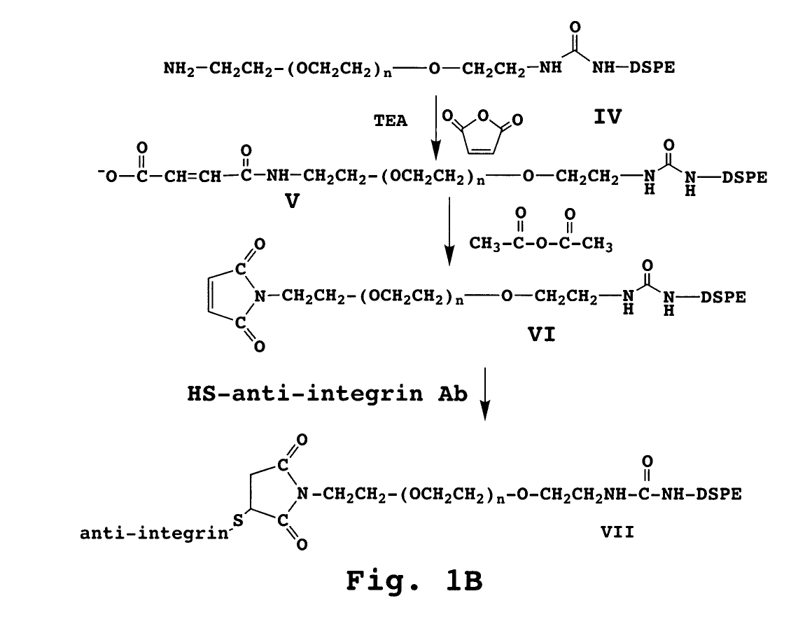

An exemplary synthetic reaction scheme is shown in FIGS. 1A-1B. Details of the reaction are given in U.S. Pat. No. 6,326,353. Briefly, polyethylene glycol (PEG) bis (amine) (Compound I) is reacted with 2-nitrobenzene sulfonyl chloride to generate the monoprotected product (Compound II). Compound II is reacted with carbonyl diimidazole in triethylamine (TEA) to form the imidazole carbamate of the mono 2-nitrobenzenesulfonamide (Compound III). Compound III is reacted with DSPE in TEA to form the derivatized PE lipid protected at one end with 2-nitrobenzyl sulfonyl chloride. The protecting group is removed by treatment with acid to give the DSPE-PEG product (Compound IX) having a terminal amine on the PEG chain. Reaction with maleic acid anhydride gives the corresponding maleamic product (Compound V), which on reaction with acetic anhydride gives the desired PE-PEG-maleimide product (Compound VI). The compound is reactive with sulfhydryl groups, for coupling the anti-integrin antibodies described herein through a thioether linkage (Compound VII).

It will be appreciated that any of the hydrophilic polymers recited above in combination with any of the vesicle-forming lipids recited above can be employed as modifying agents to prepare the lipid-polymer-ligand targeting conjugate and suitable reaction sequences for any selected polymer can be determined by those of skill in the art.

D. Liposome Preparation

Various approaches have been described for preparing liposomes having a targeting ligand attached to the distal end of liposome-attached polymer chains. One approach involves preparation of lipid vesicles which include an end-functionalized lipid-polymer derivative; that is, a lipid-polymer conjugate where the free polymer end is reactive or “activated” (see, for example, U.S. Pat. Nos. 6,326,353 and 6,132,763). Such an activated conjugate is included in the liposome composition and the activated polymer ends are reacted with a targeting ligand after liposome formation. In another approach, the lipid-polymer-ligand conjugate is included in the lipid composition at the time of liposome formation (see, for example, U.S. Pat. Nos. 6,224,903, 5,620,689). In yet another approach, a micellar solution of the lipid-polymer-ligand conjugate is incubated with a suspension of liposomes and the lipid-polymer-ligand conjugate is inserted into the pre-formed liposomes (see, for example, U.S. Pat. Nos. 6,056,973, 6,316,024).

Liposomes carrying an entrapped agent and bearing surface-bound targeting ligands, i.e., targeted, therapeutic liposomes, are prepared by any of these approaches. A preferred method of preparation is the insertion method, where pre-formed liposomes and are incubated with the targeting conjugate to achieve insertion of the targeting conjugate into the liposomal bilayers. In this approach, liposomes are prepared by a variety of techniques, such as those detailed in Szoka, F., Jr., et al., Ann. Rev. Biophys. Bioeng., 9:467 (1980), and specific examples of liposomes prepared in support of the present invention will be described below. Typically, the liposomes are multilamellar vesicles (MLVs), which can be formed by simple lipid-film hydration techniques. In this procedure, a mixture of liposome-forming lipids of the type detailed above dissolved in a suitable organic solvent is evaporated in a vessel to form a thin film, which is then covered by an aqueous medium. The lipid film hydrates to form MLVs, typically with sizes between about 0.1 to 10 microns.

The liposomes can include a vesicle-forming lipid derivatized with a hydrophilic polymer to form a surface coating of hydrophilic polymer chains on the liposomes surface. Addition of a lipid-polymer conjugate is optional, since after the insertion step, described below, the liposomes will include lipid-polymer-targeting ligand. Additional polymer chains added to the lipid mixture at the time of liposome formation and in the form of a lipid-polymer conjugate result in polymer chains extending from both the inner and outer surfaces of the liposomal lipid bilayers. Addition of a lipid-polymer conjugate at the time of liposome formation is typically achieved by including between 1-20 mole percent of the polymer-derivatized lipid with the remaining liposome forming components, e.g., vesicle-forming lipids. Exemplary methods of preparing polymer-derivatized lipids and of forming polymer-coated liposomes have been described in U.S. Pat. Nos. 5,013,556, 5,631,018 and 5,395,619, which are incorporated herein by reference. It will be appreciated that the hydrophilic polymer may be stably coupled to the lipid, or coupled through an unstable linkage, which allows the coated liposomes to shed the coating of polymer chains as they circulate in the bloodstream or in response to a stimulus.

The liposomes also include a therapeutic or diagnostic agent, and exemplary agents are provided below. The selected agent is incorporated into liposomes by standard methods, including (i) passive entrapment of a water-soluble compound by hydrating a lipid film with an aqueous solution of the agent, (ii) passive entrapment of a lipophilic compound by hydrating a lipid film containing the agent, and (iii) loading an ionizable drug against an inside/outside liposome pH gradient. Other methods, such as reverse-phase evaporation, are also suitable.

After liposome formation, the liposomes can be sized to obtain a population of liposomes having a substantially homogeneous size range, typically between about 0.01 to 0.5 microns, more preferably between 0.03-0.40 microns. One effective sizing method for REVs and MLVs involves extruding an aqueous suspension of the liposomes through a series of polycarbonate membranes having a selected uniform pore size in the range of 0.03 to 0.2 micron, typically 0.05, 0.08, 0.1, or 0.2 microns. The pore size of the membrane corresponds roughly to the largest sizes of liposomes produced by extrusion through that membrane, particularly where the preparation is extruded two or more times through the same membrane. Homogenization methods are also useful for down-sizing liposomes to sizes of 100 nm or less (Martin, F. J., in SPECIALIZED DRUG DELIVERY SYSTEMS—MANUFACTURING AND PRODUCTION TECHNOLOGY, P. Tyle, Ed., Marcel Dekker, New York, pp. 267-316 (1990)).

After formation of the liposomes, a targeting ligand is incorporated to achieve a cell-targeted, therapeutic liposome. The targeting ligand is incorporated by incubating the pre-formed liposomes with the lipid-polymer-ligand conjugate, prepared as described above. The pre-formed liposomes and the conjugate are incubated under conditions effective to association with the conjugate and the liposomes, which may include interaction of the conjugate with the outer liposome bilayer or insertion of the conjugate into the liposome bilayer. More specifically, the two components are incubated together under conditions which achieve associate of the conjugate with the liposomes in such a way that the targeting ligand is oriented outwardly from the liposome surface, and therefore available for interaction with its cognate receptor. It will be appreciated that the conditions effective to achieve such association or insertion are determined based on several variables, including, the desired rate of insertion, where a higher incubation temperature may achieve a faster rate of insertion, the temperature to which the ligand can be safely heated without affecting its activity, and to a lesser degree the phase transition temperature of the lipids and the lipid composition. It will also be appreciated that insertion can be varied by the presence of solvents, such as amphipathic solvents including polyethyleneglycol and ethanol, or detergents.

The targeting conjugate, in the form of a lipid-polymer-ligand conjugate, will typically form a solution of micelles when the conjugate is mixed with an aqueous solvent. The micellar solution of the conjugates is mixed with a suspension of pre-formed liposomes for incubation and association of the conjugate with the liposomes or insertion of the conjugate into the liposomal lipid bilayers. The incubation is effective to achieve associate or insertion of the lipid-polymer-antibody conjugate with the outer bilayer leaflet of the liposomes, to form an immunoliposome.

After preparation, the immunoliposomes preferably have a size of less than about 150 nm, preferably of between about 85-120 nm, and more preferably of between 90-110 nm, as measured, for example, by dynamic light scattering at 30° or 90°.

E. Exemplary Immunoliposomes

In studies performed in support of the invention, immunoliposomes having an antiHER2 scFv antibody were prepared as described in Example 1. In brief, liposomes were prepared from the lipids HSPC, cholesterol, and mPEG-DSPE. The therapeutic agent doxorubicin was loaded into the liposomes by remote loading against an ammonium ion gradient (Doxil®). A lipid-polymer-antibody targeting conjugate, prepared as described in Example 1 with an anti-HER2 antibody having the sequence identified herein as SEQ ID NO:2 was inserted into the pre-formed liposomes by incubation of a micellar solution containing a plurality of the conjugates with the pre-formed liposomes. Immunoliposomes having an average of 2, 5, 7.5, 15, 30, 45, 75, 100, 150, and 300 antibodies per liposome, also referred to herein as 2:1, 5:1, 7.5:1, 15:1, 30:1, 45:1, 75:1, 100:1, 150:1, and 300:1 formulations, were prepared by adjusting the reactant concentrations, as detailed in Example 1.

The in vitro uptake into HER2-expressing cells (SK-BR03) and into non-HER2 expressing cells (MCF-7) of the immunoliposomes bearing 7.5, 15, 30, and 45 antibodies was determined, as described in Example 2. The results are summarized in Table 1.

TABLE 1
Summary of Cellular Binding/Uptake of anti-HER2
Immunoliposomes in SK-BR-3 and MCF-7 Cells

| | | Doxorubicin (pg/cell) ± SE |
|---|---|---|
| Cells | Treatment | (n = 3) |
| SK-BR-3 | Control liposomes (no anti- | NS* |
| (IHC 3+) | HER2 antibody) | |
| | Anti-HER2 Immunoliposomes | 6.02 ± 0.19 |
| | (7.5:1) | |
| | Anti-HER2 Immunoliposomes | 8.35 ± 0.27 |
| | (15:1) | |
| | Anti-HER2 Immunoliposomes | 6.66 ± 0.22 |
| | (30:1) | |
| | Anti-HER2 Immunoliposomes | 4.39 ± 0.16 |
| | (45:1) | |
| MCF-7 | Control liposomes (no anti- | NS |
| | HER2 antibody) | |
| | Anti-HER2 Immunoliposomes | NS |
| | (15:1) | |
| | Anti-HER2 Immunoliposomes | NS |
| | (30:1) | |

*NS = not significant; below the detection limit

The data in Table 1 shows positive cell binding/uptake of the anti-HER2 immunoliposomes in the HER-2 positive SK-BR-3 cells, with doxorubicin levels of 2.58 pg per cell and 1.75 pg per cell for the 15:1 and 30:1 formulations, respectively. The binding/uptake of anti-HER2 immunoliposomes in the MCF-7 cells, which have low HER2 expression levels, was not significant. The binding/uptake of the liposomes not bearing anti-HER2 antibodies in both cell lines was also not significant.

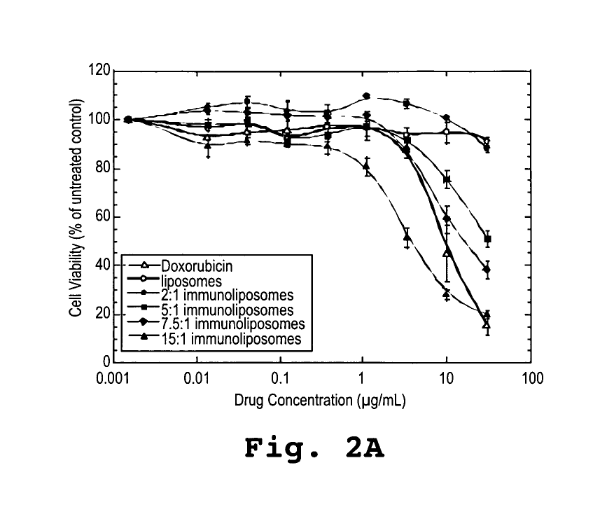

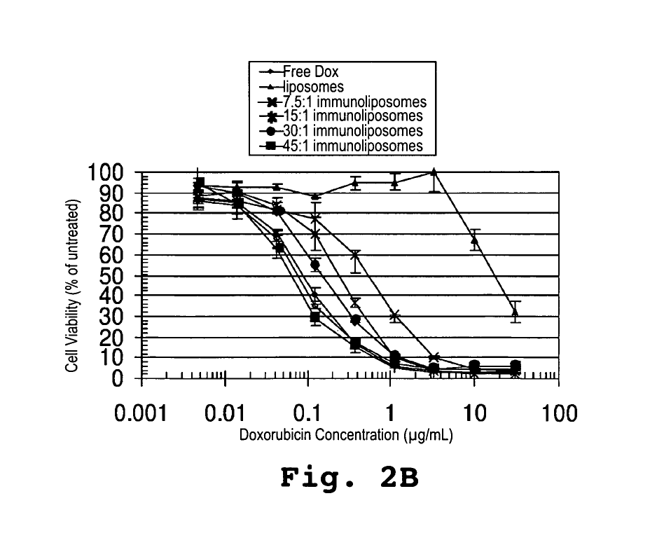

In another study, described in Example 3, the cytotoxicity of anti-HER2 immunoliposomes bearing 2, 5, 7.5, and 15 antibodies per liposome in SK-BR-3 human breast carcinoma cells was evaluated. Free doxorubicin and PEGylated, doxorubicin loaded liposomes served as positive and negative controls, respectively. The SK-BR-3 cells were exposed to anti-HER2 immunoliposomes for 10 minutes and then cell viability was evaluated. The results are shown in FIG. 2A.

FIG. 2A shows the cell viability, expressed as a percent of untreated control cells, as a function of doxorubicin concentration, in μg/mL, when administered as free drug (triangles), entrapped in liposomes (circles), or entrapped in immunoliposomes containing 2 (circles), 5 (squares), 7.5 (diamonds), or 15 (triangles) antibodies per liposome. Differences in the cytotoxicity of the liposome compositions becomes apparent at doxorubicin concentrations of about 0.5 μg/mL, where immunoliposomes decorated with 5 (squares) or 7.5 (diamonds) antibodies per liposome provided in vitro cytotoxicity roughly the same as the free drug (triangles). However, immunoliposomes with 15 antibodies per liposome (triangles) were more cytotoxic than the same concentration of free drug, with a cell viability of 50% at about 4 μg/mL doxorubicin. Immunoliposomes with 2 antibodies per liposome were less cytotoxic than the non-targeted PEGylated liposomal doxorubicin.

The cell killing effect of Her2-targeted immunoliposomes is related to the density of antibodies per liposome. IC₅₀ values for anti-Her2 immunoliposome formulations with scFv/liposome ratios of 5, 7.5, and 15 after a 10-min exposure were approximately 30, 16 and 3.9 μg/mL, respectively. Cytotoxicity for anti-Her2 immunoliposome formulations having the scFv/liposome ratio of 2 had little or no cytotoxicity after a 10 minute drug exposure. The IC₅₀ for free doxorubicin was approximately 9.5 μg/mL. Little or no cytotoxicity was noted for doxorubicin entrapped in PEG-coated liposomes. Anti-Her2 immunoliposomes with a scFv/liposome ratio of 15 had greater cytotoxicity than free doxorubicin, indicating the advantage of a targeted formulation with rapid binding followed by internalization.

Thus, in one embodiment, an immunoliposome formulation that includes an amount of lipid-polymer-antibody conjugate that provides more than 2 antibodies per liposome, and in particular, that provides on average more than 2 antibodies per liposome after circulation of the immunoliposomes in the blood in vivo for more than 24, 48, or 96 hours is contemplated, as will be discussed more below.

An alternative method of measuring in vitro cytotoxicity was performed, wherein the liposome and immunoliposome formulations were incubated with the cells for four hours. The cells were then washed and incubated at 37° C. for three days. Cell number at the end of the the three days was estimated by crystal violet staining. The results are shown in FIG. 2B. FIG. 2B shows cell viability, expressed as a percent of untreated control cells, as a function of doxorubicin concentration, in μg/mL. Four hours of exposure to various concentrations of doxorubicin from the various immunoliposomes illustrates the differing in vitro cytotoxicity of the formulations. Immunoliposomes carrying 7.5 and 45 antibodies per liposome were slightly less cytotoxic than or as cytotoxic as free doxorubicin (diamonds). Immunoliposomes with 15 and 30 antibodies were more cytotoxic than free doxorubicin. FIG. 2B also shows that immunoliposomes with 15 and 30 antibodies were more potent in cell cytotoxicity than free doxorubicin.

In another study, described in Example 4, the stability of the HER2-targeted liposomes in blood was evaluated. Immunoliposomes bearing a ¹²⁵I-labelled scFv antibody were prepared at scFv antibody/liposome ratios of 7.5, 15, 30, 45, and 90. The immunoliposomes were incubated in human plasma, and aliquots removed at selected times for analysis. Duplicate aliquots of liposomal material in human plasma were removed from incubation and applied on Sepharose CL-4B columns for each timepoint.

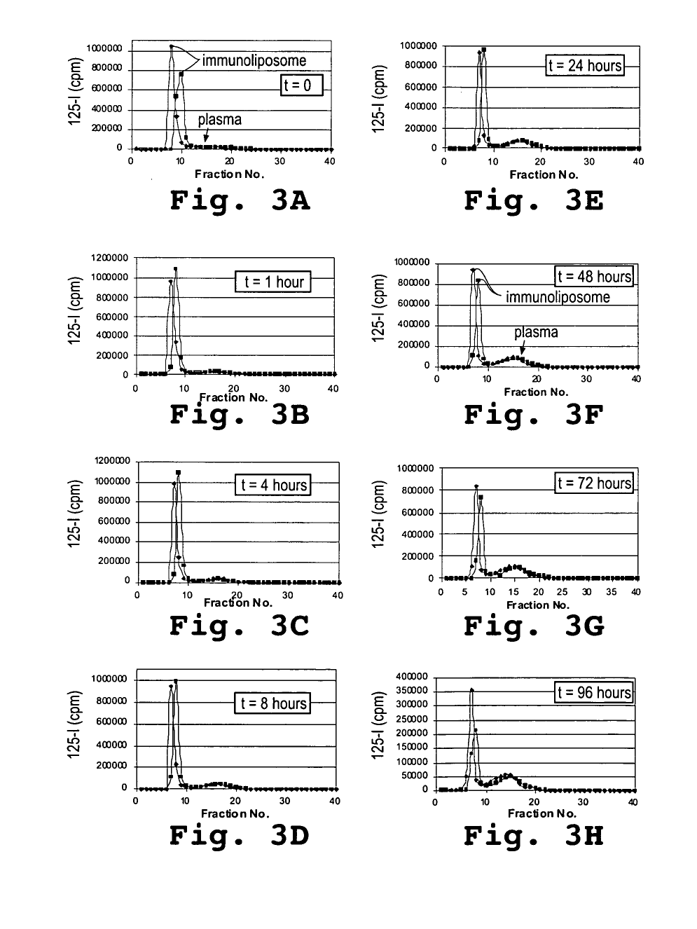

FIGS. 3A-3H show the elution profiles of ¹²⁵I-labeled lipid-PEG-scFv conjugate from immunoliposomes having 15 scFv antibodies per liposome from aliquots removed during incubation of the immunoliposomes in human plasma, the aliquots removed at times of 0 hours (FIG. 3A), 1 hour (FIG. 3B), 4 hours (FIG. 3C), 8 hours (FIG. 3D), 24 hours (FIG. 3E), 48 hours (FIG. 3F), 72 hours (FIG. 3G), and 96 hours (FIG. 3E). Radioactivity in the liposomal fraction were recovered within a very narrow range of fractions within 2-3 fractions and typically within 6-9 mL of total eluant collected. Plasma fractions, due to the wide size range of plasma proteins, eluted from the Sepharose CL-4B column over at least 12 fractions. Over all timepoints, the liposomal and plasma fraction were distinguishable from each other. In order to calculate percent of dissociated (and corresponding associated) ¹²⁵I-labeled lipid-PEG-scFv conjugate, the radioactivity from the liposomal and plasma fractions were combined to provide a total amount of ¹²⁵I-label. That total was assessed as a ratio of total radioactivity in the sample applied to the Sepharose CL-4B column.

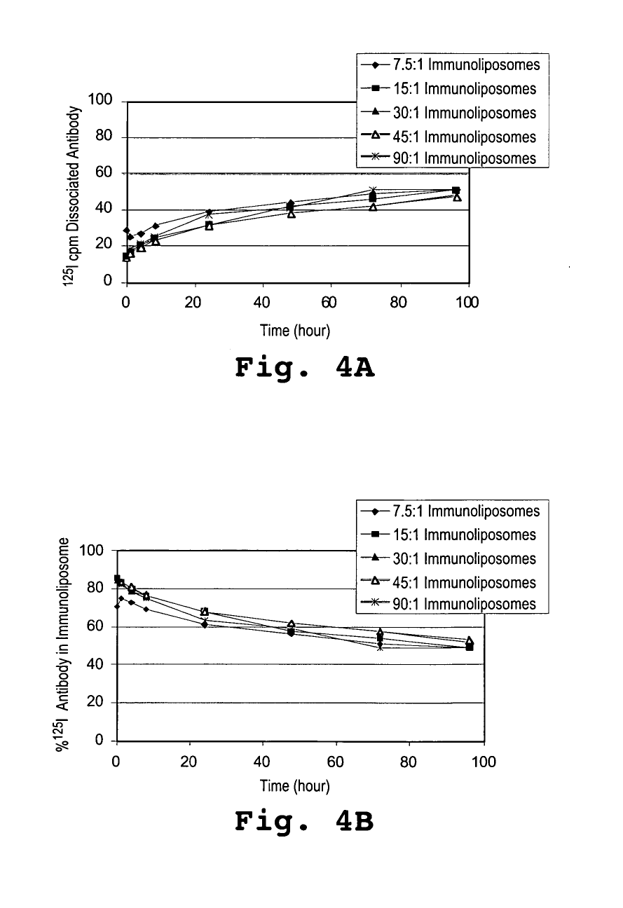

FIG. 4A shows the dissociation of ¹²⁵I-labeled scFv antibody from immunoliposome formulations having scFv antibody/liposome ratios of 7.5:1 (diamonds), 15:1 (squares), 30:1 (circles), 45:1 (triangles), and 90:1 (*) as a function of incubation time, in hours, in human plasma. FIG. 4B plots the data as percent of ¹²⁵I label remaining in the immunoliposomes for the same formulations. The rate and extent of dissociation of the antibody (relative to the amount of doxorubicin) from the formulations is essentially the same regardless of the initial density of antibodies per liposome.

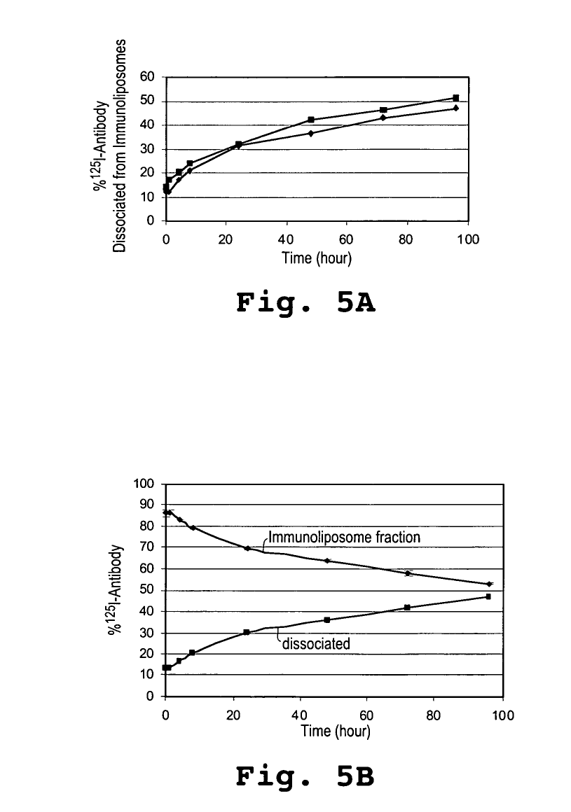

In another study, an immunoliposome formulation having 15 antibodies per liposome was prepared and the dissociation of radiolabel from the formulation during incubation in vitro in human plasma was measured. The results are shown in FIGS. 5A-5B.

FIG. 5A shows the percent of radiolabeled antibody dissociated from the immunoliposome formulation for the two studies (diamonds, squares) as a function of incubation time, in hours. The results between the two studies are consistent and indicate that 24 hours of incubation results in about 30% dissociation of the antibody from the immunoliposome. After 96 hours of incubation in human plasma, 50% of the antibodies are no longer associated with the immunoliposomes.

FIG. 5B plots the data from one of the studies using the 15:1 immunoliposome formulation as the percent of radiolabeled antibody remaining in the immunoliposomes (diamonds) and as the percent of radiolabeled antibody dissociated from the immunoliposomes (squares), as a function of incubation time in human plasma, where plasma induced dissociation is described as radioactivity recovered in the plasma fraction. FIG. 5B shows the decrease of radiolabeled scFv in the liposomal fraction with the corresponding increase in the plasma fraction expressed as a percentage of total scFv material. An initial decrease in the amount of released scFv antibody was observed reaching a steady-state by approximately 48 hours. At the time zero timepoint, 85% of the radioactivity was recovered in the liposomal fraction with the remainder in the plasma fraction. The decrease in the liposomal associated radioactivity was more pronounced within 24 hours with >30% of the scFv antibody dissociated and recovered from the plasma fraction. As the initial scFv antibody decreases in the liposomal fraction, the amount of radioactivity in the plasma fraction shows a corresponding increase (with corresponding decrease in the liposomal fraction) over the 96 hour incubation time to approximately 50% dissociation.

Accordingly, in one embodiment, an immunoliposome composition is provided that has 24 hours after in vivo administration, more preferably 48 hours after administration, and still more preferably 96 hours after administration, greater than two antibodies per liposome, on average, and fewer than 150 antibodies per liposome, on average. In another embodiment, the immunoliposome composition has an initial amount of antibodies per liposome on average, and between 30-60% of the antibodies dissociate from the immunoliposome dose administered in vivo, to provide a composition, 48 or 96 hours after in vivo administration that has fewer than about 50 antibodies per immunoliposome on average, more preferably fewer than about 30 antibodies per immunoliposome on average, and still more preferably fewer than about 15 antibodies per immunoliposome on average. In one preferred embodiment, the immunoliposomes on average include between 2 and 15, inclusive, antibodies per immunoliposome. It will be appreciated that the number of antibodies per liposome in vivo can be approximated using an in vitro assay where the immunoliposome is incubated in human plasma at 37° C. for a selected time, such as 24, 48, or 96 hours and analyzing using a suitable analytical technique for dissociation of the antibody (or the lipid-polymer-antibody construct) from the liposome.

An in vivo study was performed to evaluate the stability of the antibody in the immunoliposomes following a single intravenous administration to rats. As described in Example 5, rats were treated intravenously with either ¹²⁵I-labeled immunoliposomes (15:1 formulation) or with ¹²⁵I-labeled scFv antibody-PEG-DSPE conjugate. Blood samples were collected at 5 minutes and at 1, 3, 8, 24 and 48 hours post dosing. Whole blood and plasma samples were counted for ¹²⁵I and expressed in percent (%) of injected dose. The plasma samples from the group treated with the immunoliposomes were also assayed for doxorubicin concentration. In addition, a portion of the plasma sample at each time point was passed through a size-exclusion column to separate free conjugate from liposome-bound conjugate to determine if radioactivity was associated with liposome fraction.

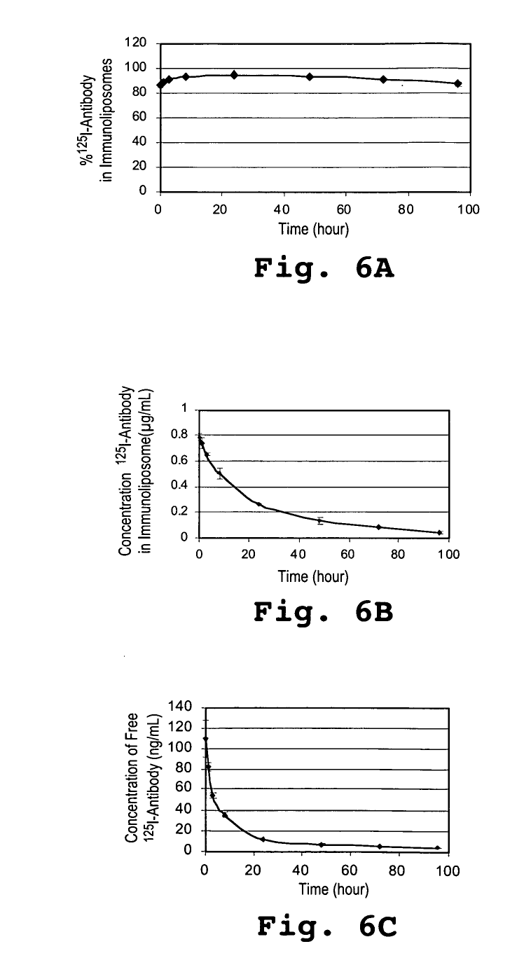

FIG. 6A shows the percentage of radiolabeled scFv antibody-PEG-DSPE conjugate recovered in the liposomal fraction of the plasma samples. Over all timepoints, >85% of the radioactivity was found only the liposomal fraction. This indicates that any free antibody or free conjugate (i.e. not liposome associated) is rapidly removed from the circulation.

FIG. 6B shows the concentration of free radiolabeled scFv antibody in the liposomal fraction of the plasma after separation on the Sepharose CL-4B column. The amount of free scFv antibody in this fraction is negligible as only small amounts (100 ng/mL and less) were recovered. Over the duration of the study, the recovery of free scFv antibody was less than 15% of the total recovered.

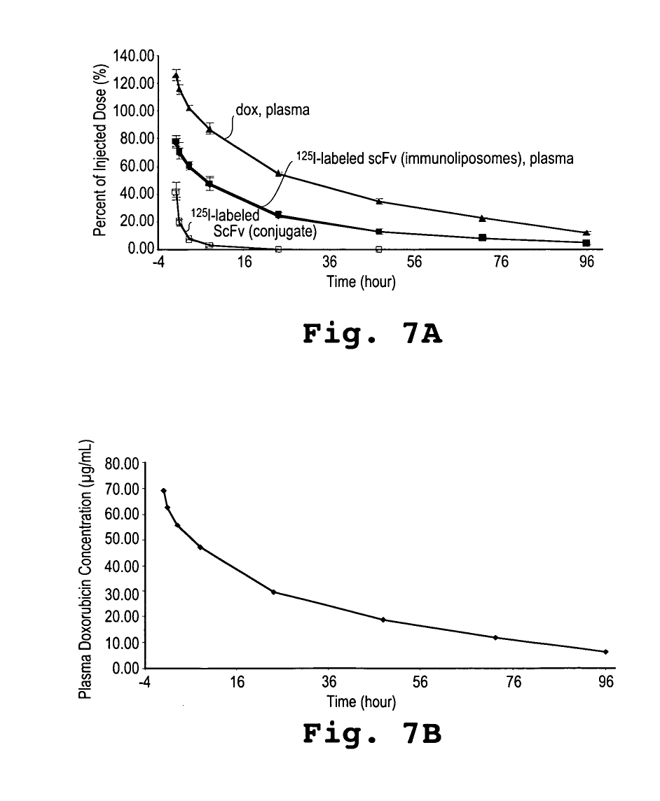

The concentration of radiolabeled scFv antibody not associated with the liposomal fraction, i.e., “free antibody” or “free conjugate” after administration of the 15:1 immunoliposome formulation to rats is shown in FIG. 6C. At the first timepoint (5 min), the initial radiolabeled scFv antibody concentration had decreased by 20% of the expected injected dose. The initial rate of radiolabeled scFv antibody elimination was rapid during the first 10 hours or so after dosing. Over the first 48 hours post dosing, the radiolabeled scFv antibody concentration decreased by 90% of the radiolabeled scFv antibody concentration at the first timepoint. In comparison 40% of doxorubicin remains in circulation (FIG. 7A).

Also as described in Example 5, the pharmacokinetic parameters of the immunoliposomes and of the doxorubicin was determined. Table 2 summarizes the pharmacokinetic parameters and FIGS. 7A-7B show the concentrations as a function of time.

TABLE 2
Pharmacokinetic Parameters of 15:1 Immunoliposome Formulation
and of scFv-antibodv-PEG-DSPE Conjugate

| | AUC^{a} | Vss^{b} | CLt^{c} | Half-Life |
|---|---|---|---|---|
| | (μg/mL*h) | (mL) | (mL/h) | (h) |
| Immunoliposome | 2224 | 13.4 | 0.31 | 31.6 |
| (DOX in plasma) | | | | |
| Immunoliposome | NA | NA | NA | 25.6 |
| (% ¹²⁵I in blood) | | | | |
| Immunoliposome | NA | NA | NA | 25.0 |
| (% ¹²⁵I in plasma) | | | | |
| Conjugate | NA | NA | NA | 2.1 |
| (% ¹²⁵I in blood) | | | | |
| Conjugate | NA | NA | NA | 2.0 |
| (% ¹²⁵I in plasma) | | | | |

^{a}Calculated using the trapezoidal method to the last time point (96 h post dose).
^{b}Volume of Distribution at steady state.
^{c}Clearance of doxorubicin from plasma

FIG. 7A shows the percentage of ¹²⁵I-labeled scFv antibody remaining in the plasma (diamonds) and in the blood (closed squares); the percentage of doxorubicin in the plasma (triangles), as a function of time, in hours, after administration of a 15:1 immunoliposome formulation to rats. Also shown is the percentage of a ¹²⁵I-labeled scFv antibody in the plasma (open circles) and in the blood (open squares) as a function of time, in hours, after administration as a free conjugate. The radioactivity in blood and plasma peaked 5 minutes post dosing both in animals dosed with immunoliposomes and in animals dosed with ¹²⁵I-labeled scFv antibody-PEG-DSPE conjugate. In animals treated with immunoliposomes, the 125I level in the blood and plasma was 78.4±4.1 and 77.2±3.7% at 5 min, and 4.4±0.9 and 4.1±0.8% at 96 h, respectively. Doxorubicin content in plasma was 127±19.7% or 69.4±10.8 μg/mL at 5 min and 11.8±3.9% or 6.4±2.2 μg/mL at 96 h. The profile of ¹²⁵I eluted from the size-exclusion column showed a single peak, which corresponded to the liposome fraction of the column effluent. Based on the percent of injected dose of ¹²⁵I in the blood and plasma, the elimination half-life of the immunoliposomes was 25.6 h and 25.0 h, respectively. Based on doxorubicin concentration in plasma, the elimination half-life of the immunoliposomes was 31.6 h.

In animals treated with ¹²⁵I-scFv antibody-PEG-DSPE conjugate, the ¹²⁵I level in the blood and plasma was 41.8±6.0 and 40.8±3.1% at 5 min, and 2.9±0.6 and 2.7±1.1% at 8 h, respectively. No radioactivity was detected at the 24 or 48 h time points. The elimination half-life of conjugate in blood and plasma was 2.1 and 2.0 h, respectively

FIG. 7B shows the plasma concentration of doxorubicin, in μg/mL, as a function of time, in hours, after administration of a 15:1 immunoliposome formulation to rats. As seen, doxorubicin remains in the blood 96 hours after dosing.

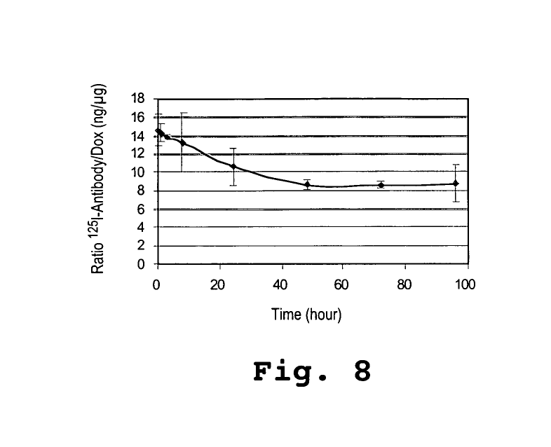

FIG. 8 is a plot of the ratio of ¹²⁵I-labeled scFv antibody to doxorubicin in the blood, in ng/μg, as a function of time, in hours, after administration of a 15:1 immunoliposome formulation to rats. The ratio of components decreases during the first 24 hours post dosing, and then plateaus at about 50 hours post dosing. The decreasing ratio shows that the scFv antibody is lost from the liposome at a faster rate than either clearance of the liposome from the circulation or loss of doxorubicin from the liposome.

In summary, the in vivo studies showed that the immunoliposomes remained in circulation for over 96 hours following a single intravenous administration in rats. The antibody-PEG-DSPE conjugate was closely associated with the liposomes as demonstrated by size-exclusion chromatography. The half-life of ¹²⁵I in blood/plasma following administration of ¹²⁵I-labeled immunoliposomes was approximately 25 hours, and the half-life of doxorubicin in plasma was 31.6 hours. When administered in free form, the antibody-PEG-DSPE conjugate remained in circulation for approximately 8 hours following a single intravenous administration. The circulation half-life of free conjugate was approximately 2 hours. The in vivo data examining the stability of the immunoliposomes in serum, showed that over all timepoints, >85% of the radioactivity remained in the liposomal fraction indicating that dissociated antibody or free antibody-conjugate is rapidly removed from the serum fraction. The immunoliposome had a measured half-life in both serum and blood of approximately 25 hours while the half-life of the free antibody or antibody-conjugate was approximately 2 hours in both blood and plasma (Table 2).

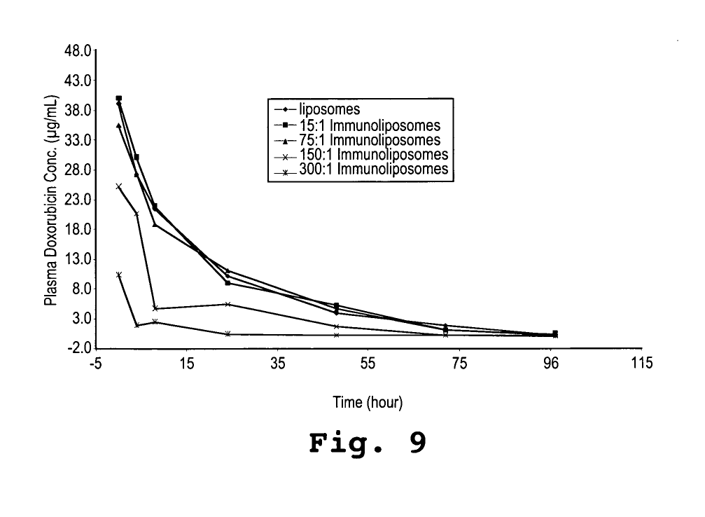

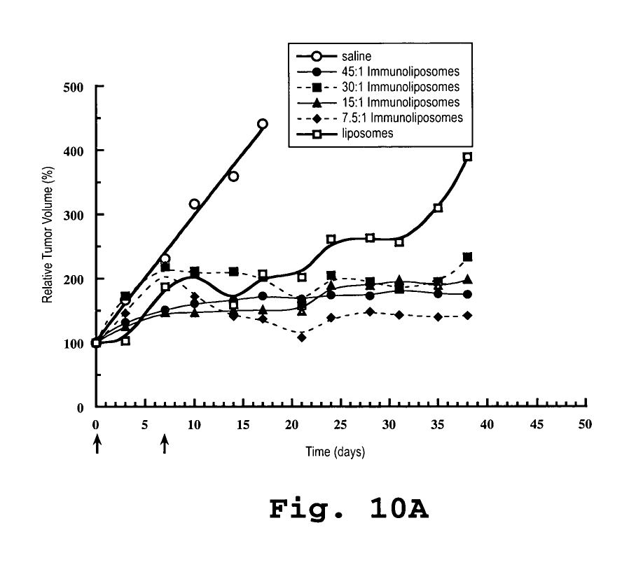

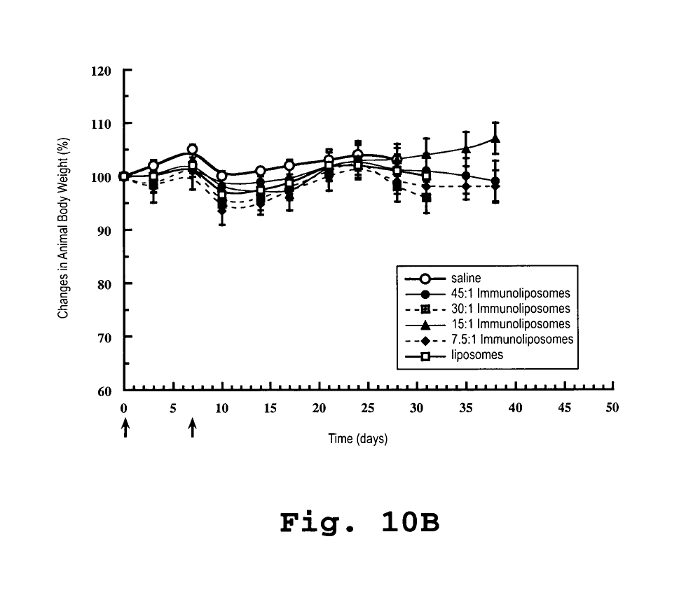

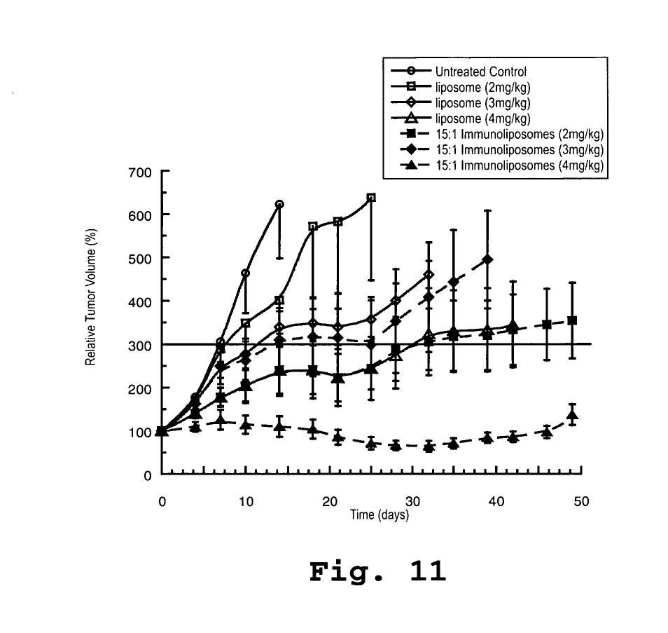

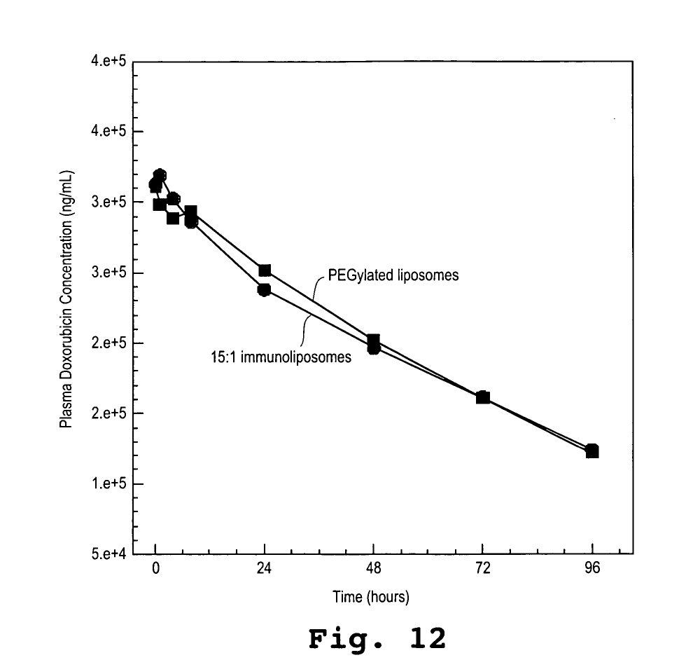

Example 6 describes another in vivo study conducted to evaluate the pharmacokinetics of immunoliposome formulations having 15, 75, 150, and 300 scFv antibodies per liposome. In brief, were treated with a single intravenous bolus of one of the immunoliposome formulations. Control mice were administered PEGylated liposomal doxorubicin. Plasma was collected at approximately 5 minutes and 4, 8, 24, 48, 72 and 96 hours post-dosing and was assayed for total doxorubicin. The results are shown in FIG. 9.

Similar doxorubicin concentration versus time profiles were observed in the animals treated with the control formulation (diamonds) and with the 15:1 (closed squares) and 75:1 (triangles) immunoliposome formulations. Considerably lower values for Cmax, AUC, and half-life were observed, accompanied by a faster clearance and a higher volume of distribution, for animals treated with the immunoliposomes having 150 (X symbols) and 300 (* symbols) antibodies per liposome.

The pharmacokinetic parameters determined from the data in FIG. 9 are shown in Table 3.

TABLE 3

| | | | | Half- | | |
|---|---|---|---|---|---|---|
| | Formulation | Cmax | AUCₗₐₛₜ^{a} | Life | Cl | Vss |
| | Group | (μg/mL) | (μg · h/mL) | (h) | (mL/h) | (mL) |
| 1 | PEGylated | 39.0 ± | 751.5 | 14.8 | 0.070 | 1.49 |
| | liposome (no | 0.27 | | | | |
| | antibody), | | | | | |
| | control | | | | | |
| 2 | 15:1 | 39.9 ± | 763.1 | 17.0 | 0.068 | 1.50 |
| | immunoliposome | 5.49 | | | | |
| 3 | 75:1 | 43.3 ± | 898.3 | 12.8 | 0.059 | 1.23 |
| | immunoliposome | 1.30 | | | | |
| 4 | 150:1 | 20.6 ± | 287.7 | 3.30 | 0.183 | 3.57 |
| | immunoliposome | 3.88 | | | | |
| 5 | 300:1 | 10.5 ± | 81.5 | 3.91 | 0.645 | 12.0 |
| | immunoliposome | 6.13 | | | | |

AUCₗₐₛₜ = the area-under-the-curve calculated to the last measured time point.

Doxorubicin concentrations in plasma peaked at the first sampling time point, i.e., approximately 5 min after injection. The Cmax values in mice administered the PEGylated liposomal control formulation, 15:1 immunoliposome formulation, and the 75:1 immunoliposome formulation were 39.0±0.27, 39.9±5.49 and 43.3±1.30 μg/mL, respectively. The corresponding drug levels decreased to 10.1±1.82, 9.05±0.69, and 13.6±5.0 μg/mL, respectively, at 24 hr, and 0.32±0.19, 0.56±0.41, and 0.28±0.12 μg/mL, respectively, at 96 hours post administration. Very similar pharmacokinetic profiles were observed in these three treatment groups. The Cmax values in mice administered 150:1 immunoliposome formulation and the 300:1 immunoliposome formulation were noticeably lower than those treated with the 75:1 or lower immunoliposome formulations, i.e., 20.6±3.88 and 10.5±6.13 μg/mL, respectively. The corresponding drug levels decreased to 4.56±0.52 and 0.58±0.19 μg/mL, respectively at 24 hr. At the end of 96 hr, half of the animals in the 150:1 and 300:1 immunoliposome formulation treatment groups had no detectable drug level in plasma.

The AUCₗₐₛₜ values in mice administered PEGylated liposomal control formulation, 15:1 immunoliposome formulation, and the 75:1 immunoliposome formulation were 751.5, 763.1 and 898.3 μg·h/mL, respectively. The AUCₗₐₛₜ values in mice administered the 150:1 and 300:1 immunoliposome formulations were 287.7 and 81.5 μg·h/mL, respectively.

Plasma half-life in mice administered PEGylated liposomal control formulation, 15:1 immunoliposome formulation, and the 75:1 immunoliposome formulation was 14.8, 17.0 and 12.8 hours respectively. Half-life in mice administered the 150:1 and 300:1 immunoliposome formulations was 3.30 and 3.91 hours, respectively.

Plasma clearance of doxorubicin in mice administered PEGylated liposomal control formulation, 15:1 immunoliposome formulation, and the 75:1 immunoliposome formulation was 0.070, 0.068 and 0.059 mL/hr, respectively. Plasma clearance of doxorubicin in mice administered 150:1 and 300:1 immunoliposome formulations was 0.183 and 0.645 mL/hr, respectively.

Volume of distribution in mice administered liposomal control formulation, 15:1 immunoliposome formulation, and the 75:1 immunoliposome formulation was 1.49, 1.50 and 1.23 mL, respectively. Volume of distribution in mice administered 150:1 and 300:1 immunoliposome formulations was 3.57 and 12.0 mL, respectively.

The findings of Example 6 show that similar pharmacokinetic profiles were observed in mice after a single bolus administration of immunoliposomes containing an scFv/liposome ratio of 0, 15, or 75. Lower values for Cmax, AUC, and half-life were observed, accompanied by a faster clearance and a higher volume of distribution, for animals treated with immunoliposomes containing an scFv/liposome ratio of 150 or 300, where the parameters were inversely proportional to the ligand density. Accordingly, in one embodiment, an immunoliposome formulation is provided that has an AUC, Cmax, and/or half-life that is (i) greater than or (ii) not more than 30%, preferably not more than 25%, still more preferably not more than 20%, lower than the than the AUC, Cmax, and/or half-life for a similar liposomal formulation lacking the targeting antibody. For example, the AUC of the control PEGylated liposome formulation lacking the antibody was 751 μg·h/mL. The AUCs of the 15:1 and 75:1 immunoliposome formulations were 763 μg·h/mL and 898 μg·h/mL, respectively. The 15:1 immunoliposome formulation had an AUC value that was 1.6% lower (i.e., not more than 25% lower) than the AUC of the corresponding liposomal formulation, identical in composition except for the absence of the antibodies. The 75:1 immunoliposome formulation had an AUC value that was greater than the AUC of the corresponding liposomal formulation. In contrast, the AUC values for the 150:1 and 300:1 immunoliposome formulations were considerably more than 30% lower than the AUC of the liposomal formulation. Similarly, the pharmacokinetic parameters of Cmax and half-life for the 75:1 and 15:1 immunoliposome formulations were more than 25% lower than the corresponding parameter of the liposomal, non-antibody targeted liposomal formulation.

Thus, the data in FIG. 9 when considered with the data in FIG. 2A and in FIG. 5B indicate that an immunoliposome formulation, and in one embodiment an immunoliposome bearing an antibody having a molecular weight of between about 20,000-50,000 Daltons, more preferably 25,000-35,000 Daltons, preferably has prior to in vivo administration between about 4-30 (inclusive of the end points) antibodies, on average, per liposome, and more preferably between about 5-25 (inclusive of the end points) antibodies per liposome, and still more preferably between about 6-20 (inclusive of the end points) antibodies per liposome. Other preferred ranges for the number of antibodies per liposome prior to in vivo administration are between about 5-30, between about 6-25, and between about 4-15, and between about 7-15.

In another embodiment, the immunoliposome formulation has, on average, more than about 2 antibodies per liposome and fewer than about 16 antibodies about 96 hours after in vivo administration, more preferably having more than about 2 antibodies per liposome and fewer than about 12 antibodies about 96 hours after in vivo administration, and still more preferably having more than about 2 antibodies per liposome and fewer than about 10 antibodies about 96 hours after in vivo administration, and even still more preferably having more than about 2 antibodies per liposome and fewer than about 8 antibodies about 96 hours after in vivo administration. Alternatively, the immunoliposome composition has more than two antibodies per liposome, on average, after about 48 hours of in vivo circulation in the blood and fewer than about 20 antibodies per liposome, on average, about 48 hours after in vivo intravenous administration.

In another embodiment, the invention provides an immunoliposome formulation having an AUC that is within 30%, preferably 25%, more preferably within 20%, of the AUC of a similar liposome formulation lacking the targeting antibodies. In another embodiment, the invention provides an immunoliposome formulation having a dose-normalized AUC that is within 30%, preferably 25%, more preferably within 20%, of the dose-normalized AUC of a similar liposome formulation lacking the targeting antibodies.

Another study was conducted to evaluate the antitumor efficacy of the immunoliposome composition. As described in Example 7, mice bearing a BT-474 human breast carcinoma xenografts, which is known to display HER2 receptor on the surface of the cells, were treated with immunoliposome formulations having no targeting antibodies (control) or having 7.5, 15, 30, or 45 antibodies per liposome. The results are shown in FIGS. 1A-10B.

FIG. 10A shows the relative tumor volume, in percent, in the mice after dosing with saline (open circles), PEGylated liposomes containing doxorubicin (open squares), or immunoliposomes containing 7.5 (diamonds), 15 (triangles), 30 (closed squares), or 45 (closed circles) scFv antibodies per liposome. Tumors in the saline control group (open circles) quadrupled in size in an average of 23.9±7.5 days. Animals treated with the immunoliposome formulations having antibody:liposome ratios of 45:1, 30:1, 15:1, and 7.5:1 had tumor volume quadrupling times of more than 35.1±2.9, 32.9±4.4, 36.1±1.9, and 38.0±0 days, respectively. Tumors in the animals treated with the control liposomal formulation (Doxil®, open squares) had tumors that quadrupled in size in more than 29.2±6.0 days. At the end of study on day 38, one animal each in the treatment groups dosed with immunoliposmes having antibody:liposome ratios of 45:1, 15:1, and 7.5:1 had either no tumor present or nodule size <10 mm³.

There were apparent tumor growth delays in all animals treated with liposomal or immunoliposomal doxorubicin when compared to saline controls. Animals treated with HER2-targeting immunoliposome formulations had slightly greater delays in tumor growth than animals treated with non-targeted liposomal doxorubicin (Doxil®).

FIG. 10B shows the percent change in body weight of the test animals during the treatment period.

In summary, the study shows that in this BT-474 human breast cancer xenograft model, treatment with liposome entrapped doxorubicin or with immunoliposome entrapped doxorubicin resulted in an enhancement in antitumor activity when compared to that of control animals. There was no difference in antitumor efficacy by varying the antibody:liposome ratios in the immunoliposome formulation from 7.5-45.

The 15:1 immunoliposome formulation was selected for evaluation in an in vivo dose range study in tumor-bearing mice. As described in Example 8, mice were treated with various dosages (2, 3, 4 mg/kg) of doxorubicin entrapped in pegylated liposomes or entrapped in immunoliposomes. All mice received a weekly intravenous dose of the appropriate formulation for three weeks. Tumor size was measured twice weekly to calculate tumor volume, and the results are shown in FIG. 11.

FIG. 11 is a plot of relative tumor volume, taken as a percent of initial tumor volume, as a function of time, in days, in mice bearing a breast tumor xenograft and treated with saline (open circles), PEGylated liposomes containing doxorubicin at dosages of 2 mg/kg (open squares) and 3 mg/kg (open diamonds) or with a 15:1 immunoliposome formulation at dosages of 2 mg/kg (closed squares), 3 mg/kg (closed diamonds) or 4 mg/kg (closed triangles). Tumors in untreated control animals (open circles) had a tumor volume tripling time (TVTT) average of 11.1±1.9 days. Animals treated with liposomal doxorubicin at 2, 3 and 4 mg/kg (open squares, diamonds, triangles, respectively) had a tumor volume tripling time (TVTT) of 16.7±4.8 days, 26.0±3.9, and >34.2±9.0 days, respectively. Animals treated with the 15:1 immunoliposome formulation TVTTs of more than 34.2±6.6, 29.5±8.6, and 49.0 for 2, 3 and 4 mg/kg dose levels, respectively.

Tumor growth delay was observed for all drug treatment groups when compared to untreated control group. There was an apparent dose response relationship for the PEGylated liposomal treatment groups but not for the immunoliposome treatment groups. However, treatment with 15:1 immunoliposome at 4 mg/kg resulted in greater delay in tumor growth than all the other treatment groups but a relationship was not evident for the immunoliposome treatment groups. Treatment with 15:1 immunoliposomes at 2 and 4 mg/kg provided mean TVTTs which were greater than the meant TVTT for the PEGylated liposomal treated groups of mice at 4 mg/kg. Further, at each dose, the mean TVTT for 15:1 immunoliposomes was greater than that for the PEGylated liposomal treated group of mice.

In another study, described in Example 9, an immunoliposome formulation containing entrapped doxorubicin and carrying 15 scFv antibodies, on average, per immunoliposome, was administered at three dosages to animals via a single intravenous injection. The pharmacokinetics of the immunoliposomes, the doxorubicin, and the antibody were determined and the results are shown in FIGS. 12-14.

FIG. 12 shows the plasma doxorubicin concentration, in ng/mL, as a function of time, in hours, after intravenous administration to monkeys of the 15:1 immunoliposome formulation (circles) and PEGylated liposomes (squares) at a doxorubicin dosage of 10 mg/mL. The blood circulation lifetime of the immunoliposomes was essentially equivalent to the PEGylated liposomes lacking the scFv antibody.

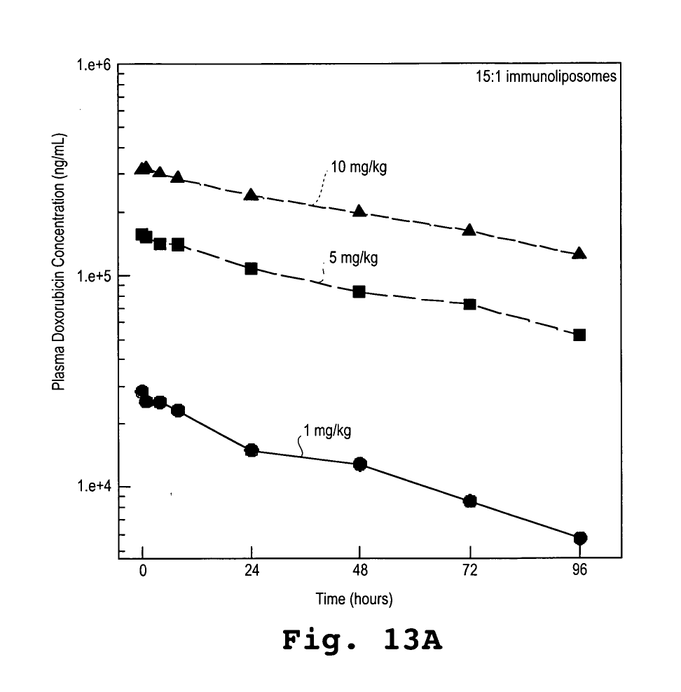

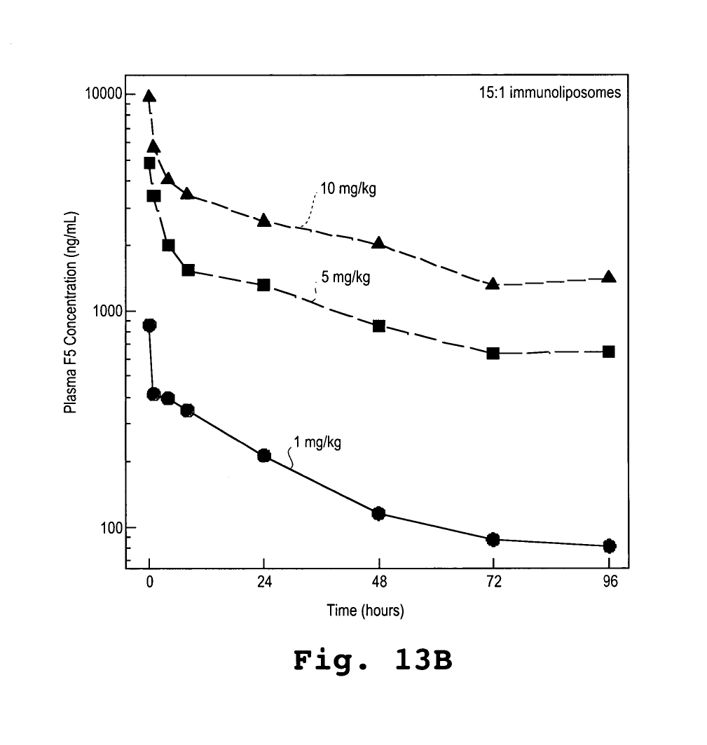

FIGS. 13A-13B show the plasma doxorubicin concentration (FIG. 13A) and the plasma antibody concentration (FIG. 13B) as a function of time after intravenous administration of the 15:1 immunoliposomes at doxorubicini dosages of 1 mg/kg (circles), 5 mg/kg (squares), and 10 mg/kg (triangles). The total (free and entrapped) doxorubicin concentration in the plasma decreases as a function of time post administration (FIG. 13A). The total (free and immunoliposome associated) antibody concentration exhibits a large initial decrease in the four hours after administration followed by a slower decline thereafter.

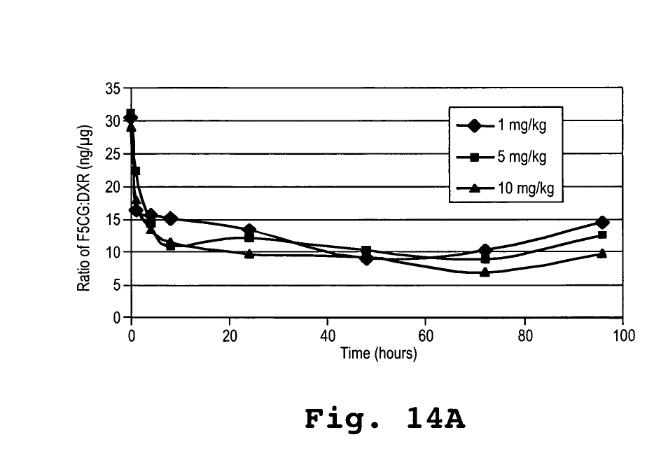

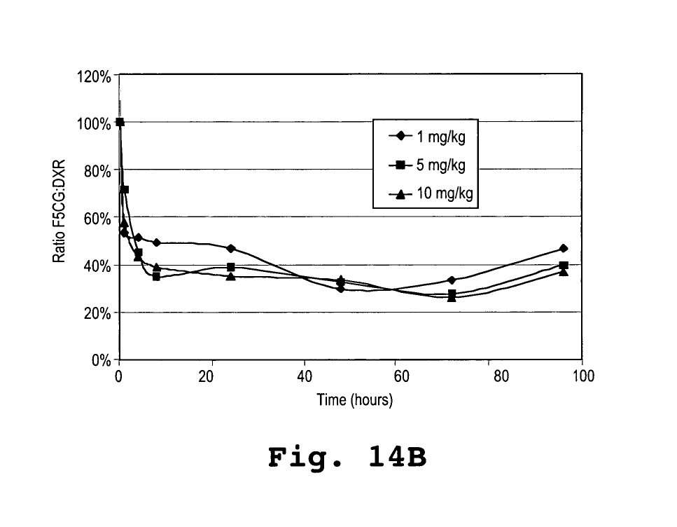

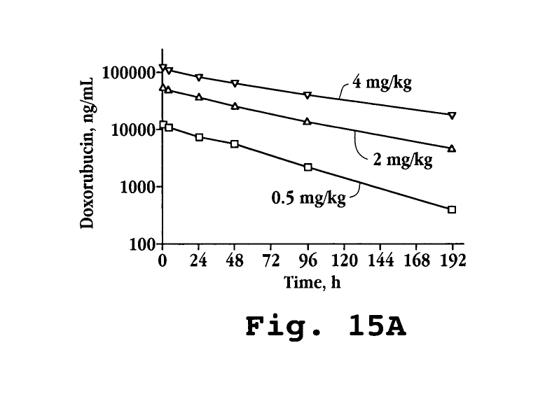

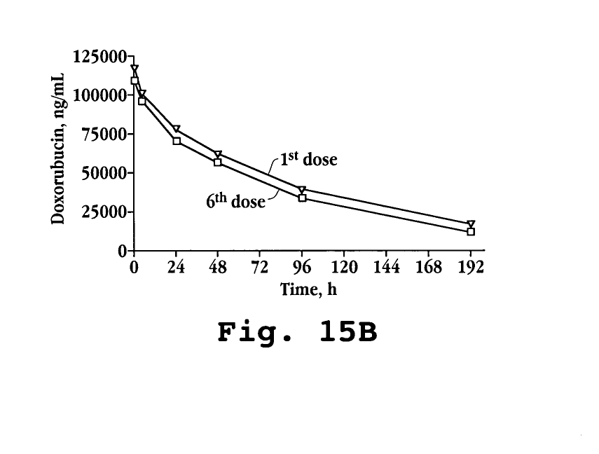

The ratio of antibody to doxorubicin concentration was determined from the data presented in FIGS. 13A-13B and is shown in FIGS. 14A-14B. In FIG. 14A, the ratio of the concentrations of scFv antibody/doxorubicin concentration in plasma, in ng/μg is shown. In FIG. 14B, the ratio is normalized to the initial antibody/doxorubicin ratio and is expressed as a percent. In both figures, the immunoliposomes administered at doxorubicin dosages of 1 mg/kg, 5 mg/kg, and 10 mg/kg are identified by diamonds, squares, and triangles, respectively. Both representations of the data illustrate the loss in antibody in the first four hours after administration, with approximately 40% of the antibody dissociated from the liposome and cleared from the bloodstream within 8 hours after dosing. It will be appreciated that the data illustrates loss of antibody, which can be a loss of the antibody from the immunoliposome or loss of lipid-polymer-antibody construct from the immunoliposome.

Thus, an immunoliposome formulation is contemplated, where the immunoliposomes lose 20-50% of the associated antibodies during in vivo circulation for a time of about 24 hours, or 48 hours, yet the immunoliposomes retain binding to the HER2 receptor sufficient for cytotoxicity. As illustrated above, greater than about two antibodies are required for in vitro cytotoxicity as compared to non-targeted liposomal doxorubicin.

Accordingly, a method of preparing an immunoliposome formulation for in vivo administration is provided, where a hydrophobic moiety-hydrophilic polymer-antibody construct is included in the formulation in a first amount sufficient to provide a first selected number of antibodies per immunoliposome. The immunoliposomes are brought in to contact with blood, in vitro or in vivo, and the number of antibodies per immunoliposome at one or more time points upon contact of the liposomes with blood is determined. For example, an aliquot of blood from the in vitro container is taken or a blood sample from an animal is drawn. The blood is analyzed for amount of antibody using any of a number of suitable analytical techniques, such as radioimmunoassay, chromatography, gel electrophoresis, etc. If the number of antibodies at more than one time point is determined, a plot can be constructed that shows the loss of antibodies as a function of time in blood. Using this information, the number of antibodies at a selected time point after contact with blood is determined and a second amount of hydrophobic moiety-hydrophilic polymer-antibody construct sufficient to provide a second, higher number of antibodies per liposome in order to provide at least two antibodies per liposome after contact with blood at such a time point is selected. The immunoliposomes are then prepared using the second amount of construct. For example, if it is desired to have 5 antibodies per immunoliposome 96 hours after administration, and the blood incubation data shows that about 50% of the antibodies dissociate from the immunoliposome or are otherwise unavailable for interaction with the target receptor, then the immunoliposomes should initially, prior to in vivo dosing, contain at least 10 antibodies. Generally, the second amount of conjugate, that is, the amount of conjugate selected to provide an initial, prior to dosing number of antibodies, is selected to yield fewer than 50 antibodies per liposome, more preferably 30 or fewer antibodies per liposome.

In another embodiment, and based in part on the pharmacokinetic data above, the method includes providing liposomes having a first amount of conjugate that provides fewer than 150 antibodies per liposome, more preferably 100 or fewer antibodies per liposome, and still more preferably 75 or fewer antibodies per liposome.

III. Methods of Use

The liposomes include a therapeutic or diagnostic agent in entrapped form. Entrapped is intended to include encapsulation of an agent in the aqueous core and aqueous spaces of liposomes as well as entrapment of an agent in the lipid bilayer(s) of the liposomes. Agents contemplated for use in the composition of the invention are widely varied, and examples of agents suitable for therapeutic and diagnostic applications are given below.

The dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. The dosage can be a one-time or a periodic dosage given at a selected interval of hours, days, or weeks.

Any route of administration is suitable, with intravenous and other parenteral modes being preferred.

In another aspect, a combined treatment regimen is contemplated, where the immunoliposome composition described above is administered in combination with a second agent. The second agent can be any therapeutic agent, including other drug compounds as well as biological agents, such as peptides, antibodies, and the like. The second agent can be administered simultaneously with or sequential to administration of the immunoliposomes, by the same or a different route of administration. Particularly preferred combinations include, but are not limited to, cyclophosphamide, taxanes, vinorelbine, Herceptin®, and Avastin®. Her2-targeted liposomes provide a more complete tumor regression relative to an equivalent dose of the same drug administered in a non-targeted liposomes, such as DOXIL®. Accordingly, a treatment regimen is contemplated that comprises a dose of a first cytotoxic agent entrapped in Her2-targeted immunoliposomes and a second therapeutic agent, wherein one or both of the agents are administered at a dose less than the dose required for the agent administered alone, to achieve an improved clinical outcome, such as a greater than expected tumor regression. It will also be appreciated that the Her2-targeted immunoliposomes can be administered in combination with two or more agents. Suitable agents for a particular patient can be identified by a skilled medical caregiver. A treatment regimen comprised of a Her2-targeted immunoliposome composition containing a chemotherapeutic agent entrapped in the liposomes, and two or more additional agents is contemplated, where at least one, and preferably more than one, of the agents is administered at dose less than the recommended dose of the agent when given alone, to achieve an improved clinical outcome.

The treatment methods described herein are intended, in one embodiment, for administration to a patient population expressing greater than 10⁵ HER2 receptors per cell, and preferably greater than 10⁶ HER2 receptors per cell. The density of cell receptors can be determined using immunohistochemistry and kits can be purchased for such determination, such as the HercepTest®. Subjects expressing greater than 10⁵, preferably greater than 10⁶ HER2 receptors per cell, respond particularly well to the HER2-targeted immunoliposome composition described herein, because the antibody described herein is internalized by the cells, increasing the drug delivered intracellularly. A method wherein a biopsy or appropriate biological sample is obtained from a patient, the sample is assayed to determine the number of HER2 receptors per tumor cell, and if the number of receptors is greater than 10⁶ HER2 receptors per cell the patient is treated with the immunoliposome composition described herein. In one preferred embodiment, the patient has an IHC score of 3+(2,000,000) receptors per cell.

IV. Examples

The following examples further illustrate the invention described herein and are in no way intended to limit the scope of the invention.

Materials: Hydrogenated soy phosphatidylcholine (HSPC) was purchased from lipoid K. G. (Ludwigshafen, Germany). Cholesterol was received from Croda, Inc. (New York, N.Y.) and N-(carbonyl-methoxypolyethylene glycol 2000)-1,2-distearoyl-sn-glycero-3-phosphatidylethanolamine, sodium salt (mPEG-DSPE) was received from Syngena, Ltd. (Liestal, Switzerland). Doxorubicin hydrochloride was received from Meiji Seika Kaisha Ltd. (Tokyo, Japan). Radiolabeled conjugate (scFv-PEG-DSPE) was received from Hermes Bioscience (San Francisco, Calif.) at a specific activity of 0.0983 mCi/mL. Human plasma was received from Bioreclamation (East Meadow, N.Y.). Column component—Sepharose CL-4B was received from Amersham Pharmacia, Uppsala, Sweden) Elution buffer—sodium chloride solution (0.9% NaCl) was from Baxter (Deerfield, Ill.) and contained 0.4% sodium azide (Sigma, St Louis, Mo).

### Example 1

### Preparation of HER2-Targeted Immunoliposomes

Liposomes containing entrapped doxorubicin were obtained from Alza Corporation Mountain View, Calif. (DOXIL®). The liposomes were composed of hydrogenated soy phosphatidylcholine (HSPC, 56.4 mole %), cholesterol (38.3 mole %), and methoxypolyethyleneglycol-di-stearoyl-phosphatidylethanolamine (mPEG-DSPE, 5.3 mole %, mPEG MW 2000 Da). The concentration of doxorubicin in the final preparation was 100 μg/mM lipid. The internal buffer used for the preparation was 10% sucrose and the external buffer was 10% sucrose and 10 mM histidine. The average diameter of liposomes in the final formulation was 93 nm.

The anti-HER2 receptor scFv antibody (SEQ ID NO:2) was first conjugated to a maleimide-derivatized PEGylated phospholipid (mPEG-DSPE) to form a lipid-PEG-scFv conjugate, according to procedures well known in the art and briefly discussed above and illustrated in FIGS. 1A-1B.

The lipid-PEG-anti-HER2 antibody construct was then associated with liposomal bilayers of the doxorubicin-loaded liposomes by incubation of the liposomes with a micellar suspension of the lipid-polymer-antibody construct. Immunoliposomes bearing on average 2, 5, 7.5, 15, 30, 45, 75, 100, 150, and 300 antibodies were prepared by adjusting the amount of antibody added per mole of phospholipid, as summarized in Table A.

TABLE A

| scFv | Amount scFv antibody |
|---|---|
| antibody:liposome | per μmole phospholipid |
| Ratio | (μg) |
| 150 | 60 |
| 100 | 40 |
| 75 | 30 |
| 45 | 18 |
| 30 | 12 |
| 15 | 6 |
| 7.5 | 3 |
| 5 | 2 |
| 2 | 0.8 |

The theoretical average number of antibodies per immunoliposome was confirmed by determining protein and phophoslipid concentrations and calculating the number of antibodies, based on an average of 80,000 phospholipid per 100 nm liposome and an antibody molecular weight of 28.714 kDa. The number of antibodies in an immunoliposome can be determined post-formation of the immunoliposomes by determining the antibody molecular weight, the size of the liposome, and the composition of the liposome. By way of example, for the 15:1 immunoliposome formulation, 15 moles of scFv antibody/80,000 moles of phospholipid×28.714 kDa=5.4 g/mol. Assuming that 90% of conjugate inserted or associated with liposomes during incubation: 5.4/90%=6 μg scFv antibody added per μmol phospholipid. Thus, the number of antibodies per immunoliposome reported herein reflect an average distribution of antibodies in the population of liposomes, with the reported number the mean number of antibodies per liposome in the population.

After association of the lipid-polymer-antibody conjugates with the liposomes, the doxorubicin concentration was 86-92 μg/mM lipid and the average diameter of immunoliposomes after was 93-117 nm.

### Example 2

### In Vitro Uptake of Immunoliposomes

SK-BR-3 and MCF7 cells were purchased from American Type Culture Collection (ATCC, Manassas, Va.). SK-BR-3 cells were maintained in in vitro culture in McCoy's 5A modified medium supplemented with 10% fetal bovine serum. MCF7 cells were maintained in Dulbecco's Modified Eagles' medium with 10% fetal bovine serum.

SK-BR-3 or MCF-7 cells at 1.5×10⁵ cells/0.5 mL of growth medium per well were added to a 24-well plate. After overnight incubation for attachment and acclimation, cells were treated with immunoliposomes (7:5:1, 15:1, 30:1, and 45:1 anti-HER2 antibodies:liposome), or liposomes at 0.015 mg/mL in 0.5 mL growth media/well in duplicates. The 24-well plates were then placed on a rotating platform inside the incubator with rotation at 40-60 rpm at 37° C., 5% CO₂, and 100% humidity for 4 hours. After incubation, cell medium were aspirated and cells were washed four times with Hank's Balanced Salt Solution (HBSS). After this, cells were lysed by adding 0.1 mL of 1% Triton X-100 to each well. The plates were swirled to mix and placed on a rotating platform for 15 minutes at room temperature. During this step, cells detached from the bottom of the plate and the cell nuclei formed visible clumps. Acid isopropanol (1.0 mL) were added to the wells containing the Triton X-100 solution and mixed by swirling or pipetting up and down until the visible clumps disappeared.

Doxorubicin content in the cell lysates was measured by a spectrofluorometer using an excitation wavelength of 470 nm and an emission wavelength of 590 nm. The spectrofluorometer settings were as follows: slit width 2 nm, integration time 1 second, photomultiplier voltage 950 V, and single wavelength acquisition mode. Fluorescence of the background cell lysate was subtracted and the amount of doxorubicin in the cell lysates was determined from the concurrently measured standards (10, 25, 50, 100, 250, 500, 1000, and 2500 ng of doxorubicin per sample) fit to a standard curve using quadratic polynomial regression. Cell uptake of doxorubicin was determined by interpolation from the standard curve. The data are expressed in pg of doxorubicin per seeded cells and are shown in Table 1. Values below the level of detection were termed not significant (NS).

### Example 3

### In Vitro Cytotoxicity of HER2-Targeted Liposomes

SK-BR-3 human breast carcinoma cell line was purchased from American Tissue Type Culture Collection (ATCC, Manassas, Va.). Cells were maintained in in vitro culture in McCoy's 5A modified medium supplemented with 10% fatal bovine serum and were maintained in a humidified incubator at 37° C.

Cells were exposed for 10 minutes to doxorubicin (1.8 mg/mL) in free form, doxorubicin entrapped in PEGylated liposomes (2.06 mg/mL), or an immunoliposome formulation having 2 antibodies per liposome (1.86 mg/mL), 5 antibodies per liposome (2.12 mg/mL), 7.5 antibodies per liposome (1.99 mg/mL), or 15 antibodies per liposome (2 mg/mL).

Log-phase SK-BR-3 breast cancer cells were harvested using Versene (1:5000), resuspended in growth media at concentration of 5×10⁴ cells/mL. An aliquot of 0.1 mL (5×10³ cells) was added to appropriate wells of a 96-well plate. After overnight incubation for attachment, medium was removed and replaced with the test materials. An aliquot of 0.1 mL of doxorubicin, S-DOX, or STEALTH 49 formulation variants, diluted with growth medium to 0.0137, 0.0412, 0.1235, 0.37, 1.11, 3.33, 10, and 30 μg/mL, was added to each well in triplicates. Medium without test material was added to control wells. Cells were exposed to the test article at 37° C. for 10 minute. At the end of incubation, the drug containing media was aspirated and cells were washed with 0.2 mL of growth media. After washing, cells were incubated in 0.2 mL fresh medium under growth conditions for another 72 hours. At the end of incubation, the amount of viable cells was determined using the Promega CellTiter 96® Aqueous One Solution Cell Proliferation Assay (a tetrazolium based colorimetric method for determining the number of viable cells in proliferation, Madison, Wis.). Briefly, media from the wells were aspirated and replaced with 0.1 mL of fresh media and 0.02 mL of Promega (Lot No. 186817). The plates were then incubated for 3-4 hours after which absorbance was recorded at 490 nm with a microplate reader. Cell viability was calculated as % of untreated control using the formula:
% Viability=(A−A₀)/(A₁₀₀−A₀)×100%
where A is the measured absorbance, A₀ is the absorbance of the blanks, and A₁₀₀ is the absorbance of the wells with untreated cells. Percent cell viability was plotted as a function of drug concentration and IC₅₀ (concentration resulting in 50% cell growth inhibition) was determined by interpolation.

The cell viability as percentage of control for the immunoliposome formulations is summarized in FIG. 2A.

### Example 4

### In Vitro Dissociation of Her2-Targeting Moiety from Liposomes in Plasma

PEGylated liposomes containing doxorubicin, prepared as described in Example 1, were combined with sufficient ¹²⁵I-labelled lipid-PEG-scFv conjugate, prepared as described in Example 1, to generate a immunoliposomes having 7.5, 15. 30, 45, and 90 scFv antibodies per liposome. The liposomes and the conjugates were incubated at 60° C. for 1 hour to allow insertion of the conjugate into the outer bilayer of the liposomes. At the end of the incubation the solution was cooled and subsequently stored at 2-8° C. Each liposome formulation was dialyzed against 10% sucrose/10 mM histidine and measured for doxorubicin content. The liposomes in each formulation were characterized for size, doxorubicin encapsulation, percent of lipid-PEG-antibody conjugate insertion, and scFv antibody concentration, which are summarized in Table B. The final specific activity of the formulation containing 15 scFv antibodies was 28.4 μCi/mL.

TABLE B

| | | % | | | scFv |
|---|---|---|---|---|---|
| Liposome | Doxorubicin | Doxo- | Particle | % | antibody |
| Formula- | Potency | rubicin | Size (nm) | Conjugate | Conc. |
| tion | mg/mL | Encap. | 90°/30° | insertion | μg/mL |
| 90 scFv/ | 2.19 | 98 | 85/101 | 91 | 472 |
| liposome | | | | | |
| 45 scFv/ | 2.00 | 98 | 83/93 | 90 | 229 |
| liposome | | | | | |
| 30 scFv/ | 2.11 | 99 | 81/94 | 91 | 152 |
| liposome | | | | | |
| 15 scFv/ | 2.10 | 99 | 80/94 | 89 | 79 |
| liposome | | | | | |
| 7.5 scFv/ | 2.12 | 98 | 82/100 | 88 | 39 |
| liposome | | | | | |

The dissociation of the ¹²⁵I label from the liposomes, indicative of dissociation of the antibody, the conjugate, or both, was measured as follows. Human plasma was mixed with ¹²⁵I-labelled HER2-targeted immunoliposome and incubated at 37° C. over 96 hours. At each timepoint (0, 1, 4, 8, 72, 96 hours) aliquots of plasma were removed and placed onto a prepared 28×1.2 cm Sepharose CL-4B column with a bed volume of 32 mL. The Sepharose CL-4B column was pre-conditioned with 1 mL 100 mM placebo liposomes and 1 mL rat plasma. Columns were eluted with 0.9% saline containing 0.4% sodium azide. Each fraction (1 mL) was counted using a gamma counter for ¹²⁵I radioactivity.

Total activity was also determined for each aliquot and recovery from the Sepharose CL-4B column was ˜90% or greater for each sample applied. Over all timepoints, there was not less than 90% recovery of radioactivity from the column relative to the total fraction removed from the applied aliquot. Results are shown in the Table below, and elution profiles for the 15:1 immunoliposome formulation are shown in FIGS. 3A-3H. FIG. 4A shows the dissociation of ¹²⁵I label from the immunoliposome formulations as a function of time. FIG. 4B shows the percent of ¹²⁵I label remaining in the liposomes as a function of time. FIG. 5A shows the dissociation rate of ¹²⁵I label from the immunoliposome formulation having 15 antibodies per liposome in two different studies. FIG. 5B shows the plasma induced release of ¹²⁵I label from the immunoliposome formulation having 15 antibodies per liposome.

Plasma Release of ¹²⁵I F5 from Immunoliposomes
at Different F5/Liposome Ratios

| | 7.5 | 15 | 30 | 45 | 90 |
|---|---|---|---|---|---|
| Time | F5/Lipo | F5/Lipo | F5/Lipo | F5/Lipo | F5/Lipo |
| 0 | 29 | 14 | 14 | 15 | 14 |
| 1 | 25 | 17 | 17 | 16 | 18 |
| 4 | 27 | 20 | 21 | 19 | 21 |
| 8 | 31 | 24 | 23 | 23 | 25 |
| 24 | 39 | 32 | 32 | 32 | 37 |
| 48 | 44 | 42 | 38 | 38 | 41 |
| 72 | 49 | 46 | 42 | 42 | 51 |
| 96 | 51 | 51 | 48 | 47 | 51 |

### Example 5

### In Vivo Dissociation of Her2-Targeting Moiety from Liposomes in Plasma

¹²⁵I-labeled scFv antibody was prepared using Iodo-beads using known techniques. In brief, the scFv antibody (SEQ ID NO:2), Na[¹²⁵I], and Iodo-beads were combined and the reaction was allowed to continue for 20 minutes at room temperature. The reaction was quenched with thiosulfate. In order to remove the excess Na[¹²⁵I] the reacted solution was separated using a DG-10 desalting column. The material in the first peak containing the highest amount of radioactivity was pooled and assessed for protein concentration, which was determined using A280. The iodinated scFv antibody was finally passed through a 0.2 μm filter for sterility and stored at 4° C. The material was used within 1 month.

Immunoliposomes having 15 antibodies per liposome were prepared as described above by incubating preformed liposomes with the ¹²⁵I-labeled scFv antibody-PEG-lipid conjugate 60° C. for 1 hour. At the end of the incubation the solution was cooled and subsequently stored at 2-8° C. The immunoliposomes were then dialyzed against 10% sucrose/10 mM histidine and measured for doxorubicin content. Final doxorubicin content was 2.1 mg/mL. A sample of the formulation was characterized for size=96 nm, pH=6.5, % doxorubicin=99%, percent of antibody insertion=89% and antibody concentration=49 μg/mL. Final specific activity of the immunoliposomes was 0.018 mCi/mL.

Free ¹²⁵I-scFv antibody-PEG-lipid was diluted in 10% sucrose/10 mM histidine to a final concentration of 49 μg/mL. In order to provide sufficient radiolabled conjugate, cold scFv antibody-PEG-lipid conjugate was added to the solution resulting in a final specific activity of 0.5 μCi/mL

Nine male CD-1 rats (Charles River Laboratories) approximately 11 weeks old and 358-376 g in weight were used in this study. The animals were acclimated to the laboratory conditions for at least 1 week.

On Day 0, all animals were warmed in a rodent hotbox prior to dosing. Animals were manually restrained and administered a single intravenous bolus of either ¹²⁵I-labeled immunoliposomes or free ¹²⁵I-F5 conjugate via a lateral tail vein. The dose administered per rat was approximately 2 mg/kg of doxorubicin and/or 49 mg/kg of the antibody-PEG-lipid conjugate.

Body weights and clinical observations were recorded on the day of dosing (Day 0). Cage side observations were made daily for 4 days. Blood samples were collected via retro-orbital sinus under inhaled anesthesia (isofluorane/O₂) into heparinized microfuge tubes. Six rats were administered immunoliposomes and blood samples (−1.2 mL) were collected (3 animals per time point) approximately 5 min, and 1, 3, 8, 24, 48, 72, and 96 hours post dose. Three rats were administered antibody-PEG-lipid conjugate, and blood samples (˜0.6 mL) were collected approximately 5 min, and 1, 3, 8, 24, and 48 hours post dose.

An aliquot of 100 μL whole blood sample from each animal was counted for ¹²⁵I using a gamma counter. The remaining blood sample was centrifuged at 2500 RPM (−750×g) for 20 min at 4-8° C. to obtain plasma. An aliquot of plasma sample (50 or 100 μL) was retained and counted for ¹²⁵I. For rats dosed with immunoliposomes, a separate set of plasma samples was stored at −40° C. and later assayed for doxorubicin concentration using a spectrofluorometer. The rest of plasma was eluted through a size-exclusion column to separate free from the liposome-bound antibody-PEG-DSPE conjugate. The column contained Sepharose-CL-4B beads with a bed volume of approximately 22 mL. The column buffer solution (0.9% saline and 0.02% NaN₃) was gravity fed at a flow rate of approximately 0.5 mL/min.

For all samples, one major peak was observed near fractions 10-15 corresponding to scFv antibody associated with the liposomal fraction. Radioactivity recovered from fractions beyond the liposomal fraction were considered to be “free scFv antibody”. That is, micellar or monomeric scFv antibody-PEG-DSPE conjugate that may be associated with or incorporated into plasma proteins. Approximately forty fractions with 1 mL each were collected and counted for ¹²⁵I.

Table C shows the recovered concentration of antibody associated with the liposomal fraction and of the free dissociated fraction. The results are shown in FIGS. 6A-6C.

TABLE C

| | | |
|---|---|---|
| | Concentration of | Concentration of Liposome- |
| Time | Free Conjugate | Bound Conjugate |

| (h) | (ng/mL) | SD | (ng/mL) | SD |
|---|---|---|---|---|
| 0.083333 | 109.4 | 18.6 | 787.2 | 26.4 |
| 1 | 82.5 | 4.3 | 741.8 | 43.0 |
| 3 | 53.9 | 2.6 | 655.3 | 14.0 |
| 8 | 35.4 | 2.3 | 504.8 | 41.8 |
| 24 | 11.6 | 0.1 | 264.9 | 1.9 |
| 48 | 7.1 | 0.9 | 134.9 | 23.1 |
| 72 | 5.4 | 0.3 | 83.6 | 3.4 |
| 96 | 4.4 | 0.4 | 43.9 | 9.4 |

Pharmacokinetic Parameters

Whole blood and plasma samples were counted for radioactivity (CPM, counts per minute) of ¹²⁵I in a Packard Cobra 5010 Gamma Counter (Serial # 401611). The CPM was converted to percent (%) of injected dose according to the following formula,
% Inj.=[(CPM/mL observed)/((CPM/mL Inj.×Vol. Inj.)/BW)×0.065]×100
where Inj.: injected; BW: body weight; and 0.065 is used to estimate the total blood volume of an animal (i.e. 6.5% BW). To calculate the percent of injected dose in plasma, the plasma volume was estimated to be 60% (0.6) that of the blood volume.

The elimination half-life (T_{1/2β}) of ¹²⁵I in blood and plasma was calculated as the following,
T_{1/2β}=ln(2)/Kel
where ln(2)=0.693 and Kel (elimination constant)=(ln(C1)−ln(C2))/|T1−T2|, where C1 and C2 equal the mean percent of injected dose at time T1 and T2. For rats dosed with immunoliposomes, T1 and T2 were 8 h and 96 h, respectively, whereas for rats dosed with the scFv-PEG-DSPE conjugate, T1 and T2 were 5 min and 8 h, respectively.

In addition, plasma doxorubicin concentration was measured using a spectrofluorometer. Pharmacokinetic parameters, e.g., area under the curve (AUC), steady state volume of distribution (Vss), Clearance (CLt) and half-life, were calculated using the WINNONLIN Noncompartmental Analysis Program Version 4.1.

The raw data is shown in Table D. The pharmacokinetic parameters are shown in Table 2 and FIGS. 7A-7B.

TABLE D
Percent of Injected Dose of ¹²⁵I in Blood and Plasma, Percent of Injected
Dose of Doxorubicin (DOX) and Doxorubicin Concentration in Plasma

| | Conjugate | | Immunoliposome | | | |
|---|---|---|---|---|---|---|
| | Whole Blood | Conjugate | Whole Blood | Immunoliposome | Immunoliposome | Immunoliposome |
| Time (h) | (¹²⁵I, %) | Plasma (¹²⁵I, %) | (¹²⁵I, %) | Plasma (¹²⁵I, %) | Plasma (DOX, %) | Plasma (DOX, μg/mL) |
| 0.083 | 41.8 ± 6.0 | 40.8 ± 3.1 | 78.4 ± 4.1 | 77.2 ± 3.7 | 127 ± 19.7 | 69.4 ± 10.8 |
| | | | | [100%] | [100%] | |
| 1 | 19.9 ± 1.8 | 19.6 ± 2.2 | 70.8 ± 5.9 | 69.5 ± 3.6 | 116 ± 13.1 | 62.7 ± 7.3 |
| | | | | [90.0%] | [91.3%] | |
| 3 | 7.1 ± 0.5 | 7.0 ± 0.3 | 60.5 ± 2.9 | 59.6 ± 2.2 | 102 ± 4.5 | 55.8 ± 2.5 |
| | | | | [77.2%] | [80.3%] | |
| 8 | 2.9 ± 0.6 | 2.7 ± 1.1 | 47.8 ± 5.0 | 47.0 ± 4.4 | 87.2 ± 19.6 | 47.1 ± 11.0 |
| | | | | [60.9%] | [68.7%] | |
| 24 | 0.0 ± 0.0 | 0.0 ± 0.0 | 25.4 ± 1.6 | 23.8 ± 0.5 | 54.8 ± 11.8 | 29.9 ± 6.4 |
| | | | | [30.8%] | [43.1%] | |
| 48 | 0.0 ± 0.0 | 0.0 ± 0.0 | 12.8 ± 2.2 | 12.5 ± 2.4 | 34.6 ± 7.6 | 18.6 ± 4.0 |
| | | | | [16.2%] | [27.2%] | |
| 72 | na | Na | 8.4 ± 0.6 | 7.8 ± 0.2 | 21.6 ± 0.5 | 11.8 ± 0.3 |
| | | | | [10.1%] | [17.0%] | |
| 96 | na | Na | 4.4 ± 0.9 | 4.1 ± 0.8 | 11.8 ± 3.9 | 6.4 ± 2.2 |
| | | | | [5.31%] | [9.29%] | |

na = sample not taken
number in []: data normalized to % of total injected dose at the 5-min time point

### Example 6

### In Vivo Administration of Immunoliposomes to Mice

Liposomes having a coating of PEG and immunoliposomes having 15, 75, 150, and 300 scFv antibodies per liposome were prepared as described in Example 1. The formulations are summarized in Table E.

TABLE E

| | Approximate Number | Doxorubicin |
|---|---|---|
| | of scFv antibodies | Concentration |
| Formulation Group | per oiposome | (mg/mL) |
| 1 Liposome (no antibody), | 0 | 2.06 |
| Control | | |
| 2 15:1 immunoliposome | 15 | 2.00 |
| 3 75:1 immunoliposome | 75 | 2.04 |
| 4 150:1 immunoliposome | 150 | 1.68 |
| 5 300:1 immunoliposome | 300 | 2.05 |

One hundred fifteen (21 per dose group×5 dose groups plus 10 extra=115) female ICR mice were obtained from Charles River. Animals were housed in conventional plastic bottomed caging under a 12 hr/12 hr light/dark lighting schedule with food and water ad libitum. Animals were acclimated to the laboratory conditions for at least 1 week prior to the start of the study.

The day of dosing was considered Day 0. All mice used were administered a single bolus injection of either control or one of the immunoliposome test formulations described above via a lateral tail vein. Dose volumes were calculated for each individual animal and ranged from 0.24 to 0.33 mL. Mice were warmed prior to injection in a rodent hotbox. Mice administered the control liposome formulation and the 15:1 and 300:1 immunoliposome formulations were dosed at approximately 2.0 mg/kg. Mice administered the 75:1 and 150:1 immunoliposome formulations were dosed at approximately 2.40 and 1.65 mg/kg, respectively.

Blood samples (˜1 mL each) were collected from three mice per time point (5 min, 4, 8, 24, 48, 72, and 96 hr) per formulation. Blood samples were collected via the hepatic portal vein under inhaled anesthesia (oxygen/Isoflurane) into heparin-coated syringes and immediately transferred into a polypropylene eppendorph tube. Blood samples were then stored on wet ice until centrifugation at approximately 2500 CPM (˜750×g) for 10 minutes at 3-8° C. Plasma samples were collected and stored at −40° C. The plasma samples and dose solutions were assayed for total doxorubicin by LC/MS analysis.

The mean plasma doxorubicin concentrations were plotted against time (FIG. 9) and used to calculate pharmacokinetic parameters. Because the plasma concentrations are all from individual animals, the pharmacokinetic parameters were calculated using the mean plasma doxorubicin HCl concentrations and therefore no standard deviations are present for the pharmacokinetic parameters pharmacokinetic parameters were calculated using WINNONLIN version 4.0 (Pharsight Corp., Mountain View, Calif.). Pharmacokinetic parameters are shown in Table 3.

### Example 7

### In Vivo Efficacy

Liposomes having a coating of PEG and immunoliposomes having 15, 75, 150, and 300 scFv antibodies per liposome were prepared as described in Example 1.

Female NCR.nu/nu homozygous mice (Charles River Laboratories, Hollister, Calif.), approximately 4-5 weeks old, were used for the obtained. The average body weight was approximately 25 g. Animals were maintained in isolator cages on a 12-hour light-and-dark cycle. Food and water were available ad libitum.

The BT-474 human breast cells were maintained in in vitro culture (RPMI 1640 medium supplemented with 10 μg/mL bovine insulin, 300 mg/mL L-glutamine, and 10% fetal bovine serum) at 37° C. in a humidified 5% CO₂ incubator. Log-phase breast cancer cells were trypsinized and harvested from cell culture flasks to yield a final concentration of 16×10⁷ cells/mL. A subcutaneous injection was made (16×10⁶ cells in 0.1 mL) on the back of the neck area of each mouse. An estradiol pellet (0.72 mg 17β estradiol, Innovative Research of America, Sarasota, Fla.) was also implanted subcutaneously in the flank of each mouse a couple days prior to tumor cell inoculation to increase tumorigenicity. Treatment started 23 days after tumor cell inoculation when mean tumor volume reached approximately 80 mm³.

Five animals were assigned to each treatment group. Immunoliposome formulations containing various antibody:liposome ratios were diluted, as appropriate, and administered intravenously (IV) at a volume of approximately 0.2 mL into the lateral tail veins of mice restrained in a heated (40° C.) brass restrainer. Immediately prior to each injection, mice were kept warm in a well-ventilated acrylic box with a heating light bulb. Treatment with the control liposomes (Doxil®) was also included and served as a positive control. Animals treated with saline served as negative controls. The doxorubicin dose used for all treatment groups was approximately 4 mg/kg per week (qw) and treatment continued for two weeks.

Animal body weights were measured twice a week to assess drug toxicity. Animals were removed from the study and provided euthanasia if a weight loss of >15% occurred or any abnormal conditions developed. Clinical observations included behavior, activity within the cage, dehydration, and signs of pain or distress. All clinical observations were recorded in the study folder. At the end of study period, all animals were provided euthanasia by inhalation of 100% carbon dioxide according to the AVMA Panel on Euthanasia (1993).

Tumors were measured in three dimensions twice weekly for up to 38 days. Tumor volume was calculated according to the formula:
V=½×D₁×D₂×D₃;
where D₁₋₃ are perpendicular diameters measured in millimeters (mm).

Tumor volume quadrupling time (TVQT), defined as the time required for a tumor to grow to four times (4×) its initial volume (at the time of treatment), was used as study endpoint. The TVQT was determined for each treatment group and expressed in days as the mean±standard error (SE).

Statistical analysis of delay in tumor growth between the various treatment groups was performed by Student's t test.

Results are shown in FIGS. 10A-10B and summarized in Table F.

TABLE F

| | Treatment | Drug Dose | | | TVQT^{b} | Growth Delay | |
|---|---|---|---|---|---|---|---|
| | Groups | (mg/kg) | Dosing Regimen | No. Mice | (M ± SE, in days) | (days) | No.^{c} Cures |
| 1 | Saline | — | Weekly ×2 | 4^{a} | 23.9 ± 7.5 | — | — |
| 2 | 45:1 immunoliposome | 4 | Weekly ×2 | 5 | >35.1 ± 2.9 | >11.2 | 1 |
| 3 | 30:1 immunoliposome | 4 | Weekly ×2 | 5 | >32.9 ± 4.4 | >9.0 | — |
| 4 | 15:1 mmunoliposome | 4 | Weekly ×2 | 5 | >36.1 ± 1.9 | >12.2 | 1 |
| 5 | 7.5:1 immunoliposome | 4 | Weekly ×2 | 5 | >38.0 ± 0 | >14.1 | 1 |
| 6 | control liposomes, | 4 | Weekly ×2 | 5 | >29.2 ± 6.0 | >5.3 | — |
| | no antibody (Doxil ®) | | | | | | |

^{a}One animal in Group 1 was excluded from the study due to tumor regression.
^{b}If any individual tumor in a treatment group had not reached its 4× volume by the end of the 38-day study period, day 38 was used in the calculation, and a ‘>’ sign was noted for that group.
^{c}Tumor size <10 mm³ as of day 38.

### Example 8

In Vivo Dose Ranging Study

Female athymic nu/nu mice (Harlan Laboratories, Ind.), approximately 4-5 weeks old, were used for the study. The average body weight was approximately 20 g. Animals were maintained in isolator cages on a 12-hour light-and-dark cycle. Food and water were available ad libitum.

The BT-474 human breast cells were maintained in in vitro culture (RPMI 1640 medium supplemented with 10 μg/mL bovine insulin, 300 mg/mL L-glutamine, and 10% fetal bovine serum) at 37° C. in a humidified 5% CO₂ incubator. Log-phase breast cancer cells were trypsinized and harvested from cell culture flasks to yield a final concentration of 15×10⁷ cells/mL. A subcutaneous injection was made (30×10⁶ cells in 0.2 mL) on the back of the neck area of each mouse. An estradiol pellet (0.72 mg 17β estradiol, Innovative Research of America, Sarasota, Fla.) was also implanted subcutaneously in the flank of each mouse a couple days prior to tumor cell inoculation to increase tumorigenicity. Treatment started 17 days after tumor cell inoculation when the mean tumor volume for all treatment groups ranged from 106 to 126 mm³.

Treatment groups are summarized in Table G. Seven animals were assigned to each treatment group. Liposome and immunoliposome formulations were administered intravenously (IV) into the lateral tail veins of mice restrained in a heated (40° C.) brass restrainer in the volume of approximately 0.2 mL. Immediately prior to each injection, mice were kept warm in a well-ventilated acrylic box with a heating light bulb. Doxorubicin dose used for the liposome formulation and the immunoliposome formulations was 2, 3, or 4 mg/kg. All treatments continued once weekly for three weeks.

TABLE G

| | | Drug Dose | | Route | | TVTT^{a} | Growth Delay |
|---|---|---|---|---|---|---|---|
| | Treatment Groups | (mg/kg) | Dosing Regimen | of Inj. | No. Mice | (M ± SE, in days) | (days) |
| 1 | Saline | — | — | — | 7 | 11.1 ± 1.9 | — |
| 2 | control liposomes, | 2 | Weekly ×3 | IV | 7 | 16.7 ± 4.8 | 5.6 |
| | no antibody (Doxil ®) | | | | | | |
| 3 | control liposomes, | 3 | Weekly ×3 | IV | 6 | 26.0 ± 3.9 | 14.9 |
| | no antibody (Doxil ®) | | | | | | |
| 4 | control liposomes, | 4 | Weekly ×3 | IV | 7 | >34.2 ± 9.0 | >23.2 |
| | no antibody (Doxil ®) | | | | | | |
| 5 | 15:1 immunoliposomes | 2 | Weekly ×3 | IV | 7 | >36.1 ± 6.6 | >25.0 |
| 6 | 15:1 immunoliposomes | 3 | Weekly ×3 | IV | 5 | >29.5 ± 8.6 | >18.4 |
| 7 | 15:1 immunoliposomes | 4 | Weekly ×3 | IV | 6 | >49.0 | >37.9 |

^{a}Tumor volume tripling time

Tumors were measured in three dimensions twice weekly for up to 49 days.

Tumor volume was calculated according to the formula:
V=½×D₁×D₂×D₃;
where D₁₋₃ are perpendicular diameters measured in millimeters (mm).

Tumor volume tripling time (TVTT), defined as the time required for a tumor to grow to three times (3×) its initial volume (at the time of treatment), was used as study endpoint. The tumor volume tripling time was determined for each treatment group and expressed in days as the mean±standard error (SE). Animal body weights were measured two times a week to assess drug toxicity.

Statistical analysis of delay in tumor growth between the various treatment groups was performed by Student's t test. The results are shown in FIG. 11.

### Example 9

### In Vivo Pharmacokinetic and Toxicity Study

Thirty-eight naïve cynomolgus monkeys (Macaca fascicularis), 3 to 8 years of age and weighing 2.5 to 4.5 kg, were randomized to six treatment groups, with 3 males and 3 females per group, summarized in Table H.

TABLE H

| | | No. | | | Dose |
|---|---|---|---|---|---|
| | | Animals | Dose | Dose Conc. | Volume |
| | Group | (M/F) | (mg/kg) | (mg/mL) | (mL/kg) |
| 1 | Saline | 3/3 | 0 | 0 | 10 |
| 2 | 15:1 immunoliposome | 3/3 | 0 | 0 | 10 |
| | placebo | | | | |
| 3 | 15:1 immunoliposome, | 3/3 | 1 | 1 | 1 |
| | Low | | | | |
| 4 | 15:1 immunoliposome, | 3/3 | 5 | 1 | 5 |
| | Mid | | | | |
| 5 | 15:1 immunoliposome, | 3/3 | 10 | 1 | 10 |
| | High | | | | |
| 6 | PEGylated liposome | 3/3 | 10 | 2 | 5 |
| | (Doxil ®, comparator) | | | | |

Immunoliposomes having 15 single chain anti-HER2 antibodies, on average, were prepared as described above. On Day 1, animals were given a single slow intravenous injection (˜1 mL/min) in a peripheral vein, and monitored for 28 days post-dose. Clinical observations and food consumption monitoring were performed at least once daily. Body weights were measured twice weekly. Blood was collected for hematology and serum chemistry analysis pre-dose and on Days 1 (1 hour post-dose), 4, 14, and 28. Blood was collected for toxicokinetic analysis at pre-dose and 5 min, 1, 4, 8, 24, 48, 72, and 96 hours following completion of dosing. The blood samples were analyzed for plasma doxorubicin and antibody concentration. The results are shown in FIGS. 12-14.

### Example 10

### In Vivo Repeat Dose Toxicity Study

Seventy naïve cynomolgus monkeys (Macaca fascicularis), 3 to 8 years of age and weighing 2.5 to 4.5 kg, were randomized to seven treatment groups, with 5 males and 5 females per group, summarized in Table I.

TABLE I

| | | | | | Cumu- |
|---|---|---|---|---|---|
| | | No. | | | lative |
| | | Animals | Dose | Dose Conc. | Dose |
| | Group | (M/F) | (mg/kg) | (mg/m²) | (mg/m²) |
| 1 | Saline | 5/5 | 0 | 0 | 0 |
| 2 | 15:1 immunoliposome | 5/5 | 0 | 0 | 0 |
| | placebo | | | | |
| 3 | 15:1 immunoliposome, | 5/5 | 0.5 | 6 | 36 |
| | Low | | | | |
| 4 | 15:1 immunoliposome, | 5/5 | 2 | 24 | 144 |
| | Mid | | | | |
| 5 | 15:1 immunoliposome, | 5/5 | 4 | 48 | 288 |
| | High | | | | |
| 6 | PEGylated liposome | 5/5 | 4 | 48 | 288 |
| | (Doxil ®, | | | | |
| | comparator) | | | | |
| 7 | doxorubicin | 5/5 | 4 | 48 | 288 |
| | (free drug) | | | | |

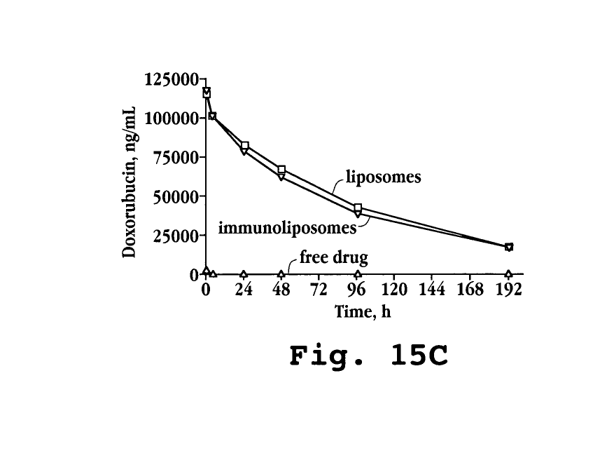

Immunoliposomes having 15 single chain antibodies with binding affinity for the HER2 cell receptor, on average, were prepared as described above. The animals were given the indicated dose intravenously once every four weeks for a total of six treatments. Blood was collected for hematology and serum chemistry analysis pre-dose and on selected time points. The blood samples were analyzed for plasma doxorubicin and the results are shown in FIGS. 15A-15C.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A composition, comprising
liposomes comprised of (i) vesicle-forming lipids; (ii) a lipopolymer; (iii) a conjugate comprised of a hydrophobic moiety, a hydrophilic polymer, and an antibody that specifically binds an extracellular domain of a HER2 receptor; and (iv) an entrapped drug, said conjugate present in an amount effective to provide, on average, greater than about 2 and fewer than about 25 antibodies per liposome.

2. The composition of claim 1, wherein said antibody has a molecular weight of between 20,000-50,000 Daltons.

3. The composition of claim 1, wherein said antibody has at least about 80% sequence identity with SEQ ID NO:2.

4. The composition of claim 1, wherein said hydrophilic polymer is polyethylene glycol having a molecular weight of between 750-5000 Daltons.

5. The composition of claim 1, wherein said entrapped drug is a cytotoxic drug.

6. The composition of claim 1, wherein said entrapped drug is an anti-tumor agent.

7. The composition of claim 1, wherein said entrapped drug is an anthracycline.

8. The composition of claim 7, wherein said drug is doxorubicin.

9. The composition of claim 1, wherein said amount of conjugate provides fewer than about 20 antibodies per liposome, on average, 48 hours after in vivo administration.

10. An immunoliposome formulation, comprising
liposomes comprised of (i) at least one rigid vesicle-forming lipid; (ii) a lipopolymer comprised of a hydrophobic moiety and polyethylene glycol; (iii) a conjugate comprised of a hydrophobic moiety, polyethylene glycol, and a single chain antibody that specifically binds an extracellular domain of a HER2 receptor, said single chain antibody having the identity of SEQ ID NO:2 or conservative substitutions thereto; and (iv) an entrapped drug having anti-tumor activity,
wherein said liposomes are characterized by an amount of conjugate effective to provide greater than about 2 and fewer than about 15 antibodies per liposome 96 hours after in vivo administration, as evidenced by an in vitro assay where the liposomes are incubated at 37° C. for 96 hours and dissociation of the single chain antibody from the liposome is determined.

11. The formulation of claim 10, wherein said rigid vesicle-forming lipid is hydrogenated soy phosphatidylcholine.

12. The formulation of claim 10, wherein said liposomes further comprise cholesterol.

13. The formulation of claim 10, wherein said entrapped drug is an anthracycline.

14. The formulation of claim 13, wherein said entrapped drug is doxorubicin.

15. The formulation of claim 10, wherein said amount of conjugate provides fewer than about 12 antibodies per liposome, on average, 48 hours after in vivo administration, as evidenced by an in vitro assay where the liposomes are incubated at 37° C. for 48 hours and dissociation of the single chain antibody from the liposome is determined.

16. An immunoliposome formulation, comprising
liposomes comprised of (i) at least one rigid vesicle-forming lipid; (ii) a lipopolymer comprised of a hydrophobic moiety and polyethylene glycol; (iii) a conjugate comprised of a hydrophobic moiety, polyethylene glycol, and a single chain antibody that specifically binds to an extracellular domain of a HER2 receptor, said single chain antibody having at least 80% identity to SEQ ID NO:2; and (iv) an entrapped drug having anti-tumor activity,
said immunoliposome formulation when administered in vivo providing an area under the curve that is greater than or not more than 25% lower than the area under the curve of liposomes comprised of like components but lacking said antibody.

17. The formulation of claim 16, wherein said rigid vesicle-forming lipid is hydrogenated soy phosphatidylcholine.

18. The formulation of claim 16, wherein said liposomes further comprise cholesterol.

19. The formulation of claim 16, wherein said entrapped drug is an anthracycline.

20. The formulation of claim 19, wherein said entrapped drug is doxorubicin.

21. The formulation of claim 16, wherein said amount of conjugate provides fewer than about 20 antibodies per liposome, on average, 48 hours after in vivo administration, as evidenced by an in vitro assay where the liposomes are incubated at 37° C. for 48 hours and dissociation of the single chain antibody from the liposome is determined.

22. The formulation of claim 16, wherein said amount of conjugate provides fewer than about 15 antibodies per liposome, on average, 96 hours after in vivo administration, as evidenced by an in vitro assay where the liposomes are incubated at 37° C. for 96 hours and dissociation of the single chain antibody from the liposome is determined.

23. An immunoliposome formulation, comprising
liposomes comprised of (i) at least one rigid vesicle-forming lipid; (ii) a lipopolymer comprised of a hydrophobic moiety and polyethylene glycol; (iii) a conjugate comprised of a hydrophobic moiety, polyethylene glycol, and a single chain antibody that specifically binds to an extracellular domain of a HER2 receptor and having a sequence identified as SEQ ID NO:2; and (iv) an entrapped drug having anti-tumor activity,
wherein 96 hours after administration of said liposomes, between 30-60% of antibodies dissociate from each liposome to provide a composition, 96 hours after in vivo administration, that has fewer than about 25 antibodies per liposome, as evidenced by an in vitro assay where the liposomes are incubated at 37° C. for 96 hours and dissociation of the single chain antibody from the liposome is determined.

24. A liposome composition prepared according to the process of
providing liposomes having an outer coating of hydrophilic polymer chains and an entrapped drug;
incubating said liposomes with an amount of conjugate comprised of a hydrophobic moiety, polyethylene glycol, and a single chain antibody that specifically binds to an extracellular domain of a HER2 receptor; said amount of conjugate being selected to provide more than about 2 and fewer than about 15 antibodies per liposome on average 96 hours after in vivo administration, as evidenced by an in vitro assay where the liposomes are incubated at 37° C. for 96 hours and dissociation of the single chain antibody from the liposome is determined.

25. The composition of claim 24, wherein said antibody has a sequence identified as SEQ ID NO:2.

26. The composition of claim 24, wherein said drug is doxorubicin.

27. The composition of claim 24, wherein said amount of conjugate provides between about 2-10 antibodies per liposome, on average.

28. A composition, comprising:
liposomes comprised of (i) vesicle-forming lipids; (ii) a lipopolymer; (iii) a conjugate comprised of a hydrophobic moiety, a hydrophilic polymer, and an antibody that specifically binds to an extracellular domain of a HER2 receptor; and (iv) an entrapped drug, said liposomes prior to in vivo administration having fewer than 25 antibodies per liposome, wherein after in vivo administration, said liposomes lose 20-50% of said antibodies yet retain binding to said Her-2 receptor sufficient for cytotoxicity.

29. The composition of claim 28, wherein said antibody is SEQ ID NO:2.

30. The composition of claim 28, wherein said drug is doxorubicin.

31. A method of preparing a liposome composition, comprising:
providing liposomes comprised of (i) vesicle-forming lipids; (ii) a lipopolymer; (iii) a conjugate comprised of a hydrophobic moiety, a hydrophilic polymer, and an antibody that specifically binds an extracellular domain of a HER2 receptor; said conjugate included in a first amount sufficient to provide a first selected number of antibodies per liposome; and (iv) an entrapped drug,
contacting said liposomes with blood;
determining the number of antibodies per liposome at one or more time points upon contact of said liposomes with blood;
selecting, based on said determining, a second amount of conjugate sufficient to provide a second, higher number of antibodies per liposome in order to provide at least two antibodies per liposome after contact with blood.

32. The method of claim 31, wherein said contacting includes contacting said liposomes with blood in vitro.

33. The method of claim 31, wherein said contacting includes contacting said liposomes with blood in vivo.

34. The method of claim 31, wherein said providing includes providing liposomes having a conjugate comprised of a phospholipid, a polyethylene glycol hydrophilic moiety, and a single chain antibody having a sequence identified herein as SEQ ID NO:2.

35. The method of claim 31, wherein said providing includes providing liposomes having doxorubicin as the entrapped drug.

36. The method of claim 31, wherein said selecting comprises selecting a second amount of conjugate effective to provide fewer than 50 antibodies per liposome.

37. The method of claim 31, wherein said providing includes providing liposomes having a first amount of conjugate that provides fewer than 150 antibodies per liposome.

38. The method of claim 31, wherein said selecting comprises selecting a second amount of conjugate effective to provide 30 or fewer antibodies per liposome.
